SEQUENCE LISTING (7 sequences)

SEQ ID NO: 1 - protein, 1049 aa, Bacillus megaterium [sequence omitted]

SEQ ID NO: 2 - protein, 503 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 3 - protein, 516 aa, Homo sapiens
MALSQSVPFSATELLLASAIFCLVFWVLKGLRPRVPKGLKSPPEPWGWPLLGHVLTLGKNPHLALSRMSQRYGDVLQIRIGSTPVLVLSRLDTIRQALVRQGDDFKGRPDLYTSTLITDGQSLTFSTDSGPVWAARRRLAQNALNTFSIASDPASSSSCYLEEHVSKEAKALISRLQELMAGPGHFDPYNQVVVSVANVIGAMCFGQHFPESSDEMLSLVKNTHEFVETASSGNPLDFFPILRYLPNPALQRFKAFNQRFLWFLQKTVQEHYQDFDKNSVRDITGALFKHSKKGPRASGNLIPQEKIVNLVNDIFGAGFDTVTTAISWSLMYLVTKPEIQRKIQKELDTVIGRERRPRLSDRPQLPYLEAFILETFRHSSFLPFTIPHSTTRDTTLNGFYIPKKCCVFVNQWQVNHDPELWEDPSEFRPERFLTADGTAINKPLSEKMMLFGMGKRRCIGEVLAKWEIFLFLAILLQQLEFSVPPGVKVDLTPIYGLTMKHARCEHVQARLRFSIN

SEQ ID NO: 4 - protein, 446 aa, Homo sapiens [sequence omitted]

SEQ ID NO: 5 - protein, 493 aa, Homo sapiens [sequence omitted]

SEQ ID NO: 6 - protein, 493 aa, Homo sapiens [sequence omitted]

SEQ ID NO: 7 - protein, 490 aa, Homo sapiens [sequence omitted]

(19) United States
(12) Patent Application Publication
(10) Pub. No.: US 2020/0061597 A1
(43) Pub. Date: Feb. 27, 2020

(54) MAGNETICALLY IMMOBILIZED METABOLIC ENZYMES AND COFACTOR SYSTEMS

(71) Applicant: ZYMtronix Catalytic Systems, Inc., Ithaca, NY (US)

(72) Inventors: Stephane Cedric Corgie, Ithaca, NY (US); Matthew Stephen Chun, Ithaca, NY (US); Rani Talal Brooks, Jefferson, LA (US)

(73) Assignee: ZYMtronix Catalytic Systems, Inc., Ithaca, NY (US)

(21) Appl. No.: 16/465,934

(22) PCT Filed: Nov. 28, 2017
(86) PCT No.: PCT/US2017/063542

§ 371 Date: May 31, 2019

Related U.S. Application Data
(60) Provisional application No. 62/429,765, filed on Dec. 3, 2016.

(51) Int. Cl. B01J 35/00; C12N 11/14; C12N 9/02; B01J 37/34; B01J 31/00; B01J 31/02; B01J 37/02

(52) U.S. Cl. CPC B01J 35/0033; C12N 11/14; C12N 9/0071; B82Y 25/00; B01J 31/003; B01J 31/02; B01J 37/0225; B01J 37/341

(57) ABSTRACT

The present invention provides compositions and methods for producing magnetic bionanocatalysts (BNCs) comprising metabolically self-sufficient systems of enzymes that include P450 monooxygenases or other metabolic enzymes and cofactor regeneration enzymes.

### CROSS REFERENCE TO RELATED APPLICATIONS

This application is the National Phase Application of PCT/US17/63542 filed Nov. 28, 2017 and claims the benefit of U.S. Provisional Application No. 62/429,765, filed on Dec. 3, 2016 each of which are incorporated herein by reference in their entirety.

### SEQUENCE LISTING

The instant application contains a Sequence Listing which has been filed electronically in ASCII format and is hereby incorporated by reference in its entirety. Said ASCII copy, created on Nov. 5, 2019, is named ZYM006US1_SL.txt and is 34,761 bytes in size.

### FIELD OF THE INVENTION

The present invention provides compositions and methods for producing magnetic bionanocatalysts (BNCs) comprising metabolically self-sufficient systems of enzymes that include P450 monooxygenases or other metabolic enzymes and cofactor regeneration enzymes.

### BACKGROUND OF THE INVENTION

Magnetic enzyme immobilization involves the entrapment of enzymes in mesoporous magnetic clusters that self-assemble around the enzymes. The immobilization efficiency depends on a number of factors that include the initial concentrations of enzymes and nanoparticles, the nature of the enzyme surface, the electrostatic potential of the enzymes, the nanoparticle surface, and the time of contact. Enzymes used for industrial or medical manufacturing in biocatalytic processes should be highly efficient and stable before and during the process, reusable over several biocatalytic cycles, and economical. Enzymes used for screening and testing drugs or chemicals should be stable, reliable, sensitive, economical, and compatible with high-throughput automation.

P450-generated pharmacologically active metabolites are potential resources for drug discovery and development. There are several advantages of using drug metabolites as active ingredients because they can show superior properties compared to the original drugs. This includes improved pharmacodynamics, improved pharmacokinetics, lower probability of drug-drug interactions, less variable pharmacokinetics and/or pharmacodynamics, improved overall safety profile and improved physicochemical properties.

Cytochrome P450 (referred to as P450 or CYP) are of the E.C. 1.14 class of enzymes. (Br. J. Pharmacol. 158(Suppl 1): S215-S217 (2009), incorporated by reference herein in its entirety.) They constitute a family of monoxygenases involved in the biotransformation of drugs, xenobiotics, alkanes, terpenes, and aromatic compounds. They also participate in the metabolism of chemical carcinogens and the biosynthesis of physiologically relevant compounds such as steroids, fatty acids, eicosanoids, fat-soluble vitamins, and bile acids. Furthermore, they are also involved in the degradation of xenobiotics in the environment such pesticides and other industrial organic contaminants. They function by incorporating one hydroxyl group into substrates found in many metabolic pathways. In this reaction, dioxygen is reduced to one hydroxyl group and one H₂O molecule by the concomitant oxidation of a cofactor such as NAD(P)H.

Monooxygenases are key enzymes that act as detoxifying biocatalysts in all living systems and initiate the degradation of endogenous or exogenous toxic molecules. Phase I metabolism of xenobiotics includes functionalization reactions such as oxidation, reduction, hydrolysis, hydration and dehalogenation. Cytochrome P450 monooxygenases represent the most important class of enzymes involved in 75-80% of metabolism. Other phase I enzymes include monoamine oxidases, Flavin-containing oxygenases, amidases and esterases.

Phase II metabolism involves conjugation reactions (glucuronidation, sulfation, GSH conjugation, acetylation, amino acid conjugation and methylation) of polar groups (e.g. glucuronic acid, sulfate, and amino acids) on phase I metabolites.

In recent years there has been an increasing interest in the application of P450 biocatalysts for the industrial synthesis of bulk chemicals, pharmaceuticals, agrochemicals, and food ingredients, especially when a high grade of stereo and regioselectivity is required.

P450 monooxygenase enzymes are labile and notoriously difficult to use in biocatalytic reactions. They are, however, a major component of the metabolic pathway of drug and xenobiotic conversions and hence play an important role in the generation of drug metabolites and detoxification of chemicals. There is a growing need for new ways to produce a diversity of chemical metabolites by metabolic enzymes, including P450s. They are used in drug development for pharmacokinetic and biodegradation studies of chemicals. Recombinant Cytochrome P450 BM3 (BM3) has been considered one of the most promising monoxygenases for biotechnological and chemical applications because of its high activity and ease of expression from recombinant vectors in common hosts such as B. megaterium or E. coli. BM3 are all in one catalysts as they possess the oxidative activity and a co-factor reduction activity. Structurally, the P450 domain is fused with a reductase domain to facilitate the direct transfer of electrons. Moreover, the molecules are soluble and do not have to be membrane bound. This provides advantages for production and use in biocatalytic reactions. Thus, developing novel methods for employing P450s in biocatalyst reactions is of significant commercial interest.

P450s, and most metabolic oxidative enzymes in general, require a cofactor for the conversion of their target compounds. Protons (H⁺) are usually delivered from the cofactor NADH or NADPH through specific amino acids in the CYP enzyme. They relay the protons to the active site where they reductively split an oxygen molecule so that a single atom can be added to the substrate. CYP enzymes receive electrons from a range of different redox partner enzymes including, but not limited to, glucose dehydrogenase (GDH) and formate dehydrogenase (FDH).

GDH (E.C. 1.1.1.47) catalyzes the oxidation of β-D-glucose to β-D-1,5-lactone with simultaneous reduction of NADP+ to NADPH or of NAD+ to NADH. FDH (EC 1.2.1.2) refers to a set of enzymes that catalyze the oxidation of formate to carbon dioxide. They donate electrons to a second substrate such as NAD+. These enzymes, especially from eukaryotic sources, have total-turnover numbers amongst the lowest of any enzymes. Biocatalytic reactions with cytochromes P450 are highly inefficient because substrate oxidation is associated with the production of Reactive Oxygen Species (ROS), e.g., hydrogen peroxide and superoxide, as by-products. For eukaryotic monooxygenases, a large fraction of the activated oxygen from the enzymes are diverted from the oxidation of the targets and converted to ROS by either decay of the one-electron-reduced ternary complex that produces a superoxide anion radical (O-2), while the protonation of the peroxycytochrome P450 and the four-electron reduction of oxygen produce H₂O₂. Hence, eukaryotic P450 enzymes lose a very substantial part (>30%) of the consumed reducing equivalents for the production of ROS.

Compared to eukaryotic P450, bacterial P450s are more efficient as less than 10% of the total electron intake is diverted to ROS resulting in better efficiency of O₂ and electron conversion efficiency in the oxidation route. Special designs in bioreactors are necessary to control dissolved oxygen concentrations at levels that prevent the buildup of ROS without slowing down the reactions.

Oxidative inhibition due to the production of reactive oxidative species (ROS) is one of the major limitations of P450 biocatalysis. Reactive Oxygen Species (ROS) are a major by-product of the metabolic reactions of P450s and other oxidases including NADPH Oxidase (NOX), Lipoxygenase (LOX) and cyclooxygenase (COX). Reactive oxygen species (ROS) include highly reactive oxygen radicals [superoxide (O2.-), hydroxyl (.OH), peroxyl (RO2.), alkoxyl (RO.)] and non-radicals that are either oxidizing agents and/or are easily converted into radicals. Examples include hypochlorous acid (HOCl), ozone (O₃), singlet oxygen (1O2), and hydrogen peroxide (H₂O₂) as hydrogen peroxide (H₂O₂) and superoxide ion (O₂₋) if the reaction occurs in an excess of oxygen. High levels of ROS not only reduce the efficiency of the conversion reactions but also inhibit the reactions due to oxidative denaturation. One way to prevent ROS build up during an oxidative reaction is to scavenge key intermediaries using ROS degrading enzymes such as catalases or superoxide dismutases (SOD). They decontaminate the ROS while producing dioxygen and recycle oxygen radicals that can be used for the P450 oxidation cycles.

Other metabolic enzymes known in the art that produce metabolites in Phase I, II and III metabolism include UDP-glucuronosyl transferases, sulfotransferases, flavin-containing monooxygenases, monoamine oxidases, and carboxyesterases. Metabolic enzymes have low activity and are particularly unstable ex-vivo. In order to get high and fast production of chemical metabolites for screening or in biochemical production, the concentration of P450s has historically been high (50 to 200% substrate loading). In order to increase the oxidation rate of the target compounds, oxygen levels also need to be high at over-stoichiometric concentrations. This leads to the production of superoxide anions that denature the enzymes and limit the efficiency of the reaction.

New ways to combine in defined ratios, stabilize, use and reuse metabolic enzymes such as P450s are needed to produce chemical metabolites qualitatively and quantitatively. In order to be used for the metabolic screening of thousands of chemicals, P450 and combinations of metabolic enzymes need to be conditioned in a high-throughput format that are compatible with automation. This can be achieved by performing reactions in microplates. Dioxygen can become a limiting factor affecting the yield of P450 reactions.

Increasing the diffusion of dioxygen by mixing over the course of long reactions is important to increase rates of reaction and productivity of the P450s. Stirring in a microplate format is, however, challenging due to the limited volume and number of wells. Gentle mixing increases the oxygenation of the reaction mix without damaging the materials and the enzymes is an important unmet need in the art. The sequence of incubation, mixing, and collecting supernatants should be integrated into an automated, high-throughput workflow.

### SUMMARY OF THE INVENTION

The present invention provides compositions and methods for producing bionanocatalysts (BNCs) comprising magnetically immobilized enzymes that require a diffusible cofactor combined with a cofactor regenerating enzyme. In some embodiments, the cofactor-dependent enzyme is a P450 Monooxygenase combined with a reductase. In some instances, the cofactor is co-immobilized with the enzymes to increase productivity.

Thus, the invention provides a composition comprising self-assembled mesoporous aggregates of magnetic nanoparticles and a first enzyme requiring a diffusible cofactor having a first enzymatic activity; a second enzyme comprising a cofactor regeneration activity; wherein the cofactor is utilized in the first enzymatic activity; wherein the first and second enzymes are magnetically-entrapped within the mesopores formed by the aggregates of magnetic nanoparticles and the first and second enzymes function by converting a diffusible substrate into a diffusible product.

In some embodiments, the co-factor is entrapped in the mesoporous aggregates of magnetic nanoparticles with the first and second enzymes. In other embodiments, the mesoporous aggregates of magnetic nanoparticles have an iron oxide composition. In other embodiments, the mesoporous aggregates of magnetic nanoparticles have a magnetic nanoparticle size distribution in which at least 90% of magnetic nanoparticles have a size of at least 3 nm and up to 30 nm, and an aggregated particle size distribution in which at least 90% of the mesoporous aggregates of magnetic nanoparticles have a size of at least 10 nm and up to 500 nm. In other embodiments, the mesoporous aggregates of magnetic nanoparticles possess a saturated magnetization of at least 10 emu/g. In preferred embodiments, the mesoporous aggregates of magnetic nanoparticles possess a remanent magnetization up to 5 emu/g. In other embodiments, the first and second enzymes are contained in the mesoporous aggregates of magnetic nanoparticles in up to 100% of saturation capacity.

In some embodiments of the invention, the first and second enzymes are physically inaccessible to microbes.

In some embodiments of the invention, the first enzyme is an oxidative enzyme. In preferred embodiments, the oxidative enzyme is a Flavin-containing oxygenase; wherein the composition further comprises a third enzyme having a co-factor reductase activity that is co-located with the first enzyme. In other embodiments, the oxidative enzyme is a P450 monooxygenase; wherein the composition further comprises a third enzyme having a co-factor reductase activity that is co-located with the first enzyme. In preferred embodiments, the P450 monooxygenase and the third enzyme are comprised within a single protein. In more preferred embodiments, the single protein comprises a bifunctional cytochrome P450/NADPH—P450 reductase. In more preferred embodiments, the single protein has BM3 activity and has at least a 90% sequence identity to SEQ ID NO:1. In other embodiments, the P450 has at least a 90% sequence identity to any one of SEQ ID NOS:2-7.

In some embodiments of the invention, the P450 monooxygenase is co-located with the third enzyme within a lipid membrane. In preferred embodiments, the third enzyme is a cytochrome P450 reductase.

In some embodiments, the P450 monooxygenase comprises a P450 sequence that is mammalian. In other embodiments, the P450 monooxygenase comprises a P450 sequence that is human. In other embodiments, the P450 monooxygenase comprises CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1,CYP3A4, CYP3A5, CYP3A7, CYP3A43,CYP4A11, CYP4A22, CYP4B1, CYP4F2, CYP4F3, CYP4F8, CYP4F11, CYP4F12, CYP4F22, CYP4V2, CYP4X1, CYP4Z1,CYP5A1,CYP7A1, CYP7B1,CYP8A1, CYP8B1,CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1, or CYP51A1.

In some embodiments, the P450 monooxygenase comprises a P450 sequence that is of an origin selected from the group consisting of primate, mouse, rat, dog, cat, horse, cow, sheep, and goat. In other embodiments, the P450 monooxygenase comprises a P450 sequence that is of an origin selected from the group consisting of insect, fish, fungus, yeast, protozoan, and plant.

In some embodiments, the second enzyme is selected from the group consisting of a carbonyl reductase, an aldehyde dehydrogenase, an aryl-alcohol dehydrogenase, an alcohol dehydrogenase, a pyruvate dehydrogenase, a D-1 xylose dehydrogenase, an oxoglutarate dehydrogenase, an isopropanol dehydrogenase, a glucose-6-phosphate dehydrogenase, a glucose dehydrogenase, a malate dehydrogenase, a formate dehydrogenase, a benzaldehyde dehydrogenase, a glutamate dehydrogenase, and an isocitrate dehydrogenase.

In some embodiments of the invention, the cofactor is nicotinamide adenine dinucleotide+hydrogen (NADH), nicotinamide adenine dinucleotide phosphate+hydrogen (NADPH), Flavin adenine dinucleotide+hydrogen (FADH), or glutathione.

Some embodiments of the invention further comprise a fourth enzyme that reduces a reactive oxygen species (ROS). In preferred embodiments, the fourth enzyme is a catalase, a superoxide dismutase (SOD), or a glutathione peroxidase/glutathione-disulfide reductase.

In some embodiments, the first enzyme participates in phase I metabolism. In other embodiments, the invention provides a fifth enzyme that participates in phase II or phase III metabolism. In preferred embodiments, the fifth enzyme is a UDP-glucoronosyl transferase, a sulfotransferase, a monoamine oxidase, or a carboxylesterase.

The invention provides that the composition of mesoporous aggregates may be assembled onto a macroporous magnetic scaffold. In preferred embodiments, the macroporous magnetic scaffold is a polymeric hybrid scaffold comprising a cross-linked water-insoluble polymer and an approximately uniform distribution of embedded magnetic microparticles (MMP). In preferred embodiments, the magnetic macroporous polymeric hybrid scaffold comprises PVA and a polymer selected from the group consisting of CMC, alginate, HEC, and EHEC.

The invention provides that one or more the enzymes are produced by recombinant DNA technology or cell-free protein synthesis.

The invention provides a method of manufacturing a chemical, comprising exposing the composition disclosed herein to the diffusible substrate in a first reaction.

Preferred embodiments further comprise the step of magnetically mixing the first reaction. Preferred embodiments further comprise recovering the diffusible product. Other preferred embodiments comprise magnetically recovering the composition from other components of the first reaction. More preferred embodiments comprise the step of exposing the composition to a second reaction. More preferred embodiments comprise recovering the diffusible product from the second reaction.

In some embodiments, the first reaction is a batch reaction. In preferred embodiments, the batch reaction is in a microplate. Other embodiments include a packed bed reaction or a continuous flow reaction.

### BRIEF DESCRIPTION OF THE DRAWINGS

FIG. 1. Metabolic enzymes magnetically-immobilized in a bionanocatalyst (BNC). The BNC includes immobilized \P450-BM3 (reductase fused to a monooxygenase), glucose dehydrogenase (GDH), catalase (CAT), superoxide dismutase (SOD) and an NADPH cofactor.

FIG. 2. Metabolic Phase I metabolic enzymes magnetically-immobilized in a bionanocatalyst (BNC). Human recombinant P450 monooxygenase in a vesicular membrane that includes a reductase enzyme. The BNC also includes immobilized glucose dehydrogenase (GDH), catalase (CAT), superoxide dismutase (SOD), and an NADPH cofactor.

FIG. 3. Activity and Reusability of BM3 cytochrome P450 co-immobilized with support enzymes and cofactors compared to the free enzyme systems. The BM3-p450 variant was immobilized in BNCs with 20% total protein including glucose dehydrogenase (GDH), catalase (CAT), superoxide dismutase (SOD), and NADPH. These BNCs were templated onto magnetic macroporous polymeric hybrid scaffolds forming Biomicrocatalystss (BMC) with a total protein loading of 0.5% and 0.17% P450 loading. BMCs were reused in 10 sequential p-nitrophenyl laurate oxidation assays (18 hour incubation). Free enzyme stock prepared for the immobilization was tested each day but showed no activity after 2 days.

FIGS. 4A to 4C. Bacterial growth suppression from immobilized P450. After 24 h, a liquid bacterial culture containing free BM3-variant prepared fresh from lyophilizate became turbid. A sample from the turbid stock was grown for 24 h in LB broth at 37° C., then streaked on LB agar then incubated for 24 h at 37° C. (FIG. 4A). Supernatant from immobilized BM3-P450 was similarly cultured but yielded no bacterial growth (FIG. 4B). All colonies had the same morphologies. Phase-contrast microscopy (FIG. 4C) revealed a Bacillus. These data suggest a single species and may in fact be the host used to express the recombinant P450-BM3.

FIGS. 5A-5D. Magnetic BMC mixing in a high-throughput microplate format (96 well plate). Permanent magnets moved in tandem (FIGS. 5A and 5B) above and below a stationary sealed 96-well microplate bounce BMCs in a reaction medium. For electronic mixing, alternating activation of electromagnets (FIGS. 5C and 5D) situated directly above and below a stationary sealed 96-well microplate bounce BMCs in a reaction medium.

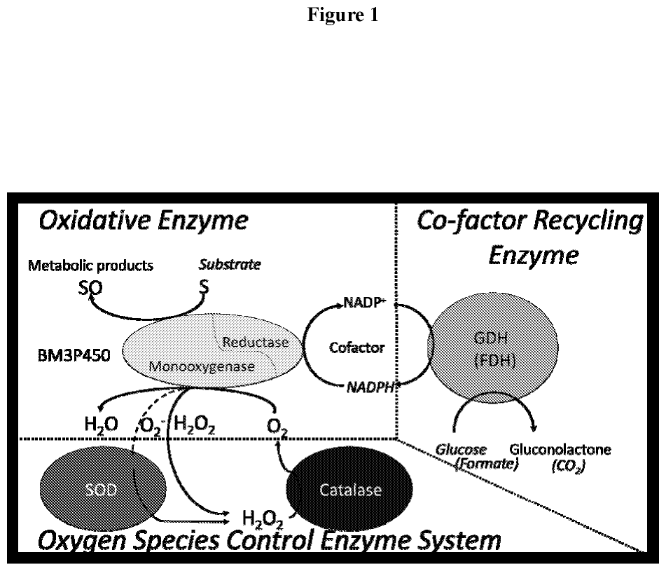

### DETAILED DESCRIPTION OF THE INVENTION

The present invention provides compositions and methods for producing and and using BNCs comprising metabolic enzymes such as P450 Monooxygenases in combination with other metabolic enzymes and supporting enzymes to enhanced metabolic performances and stability. The BNCS form by self-assembly and contain 5-20,000 micrograms of P450, or total proteins, per gram of nanoparticles. The BNCs prevent loss of enzyme activity upon immobilization, maximize enzyme loading, or allow the immobilized enzymes to be scaffolded onto magnetic materials for ease of processing with a magnetic mixing apparatus immobilizing enzymes into magnetic materials enables incubating these magnetic biocatalysts in a microplate format in a magnetic mixer and using the magnetic material as the stirring component of the reaction. At the end of the reaction, the materials can be captured at the bottom of the plate so that the supernatant containing the compounds of interest can be retrieved. Applied to the larger scale production of metabolites, the magnetic materials allow to recycle the enzymes for subsequent or continuous reactions.

Self-assembled mesoporous nanoclusters comprising magnetically-immobilized enzymes are highly active and stable prior to and during use. Magnetically immobilized enzymes do not require bonding agents for incorporation into the self-assembled mesopores formed by the magnetic nanoparticles (MNPs). No permanent chemical modifications or crosslinking of the enzymes to the MNPs are required. The technology is a blend of biochemistry, nanotechnology, and bioengineering at three integrated levels of organization: Level 1 is the self-assembly of enzymes with MNP for the synthesis of magnetic mesoporous nanoclusters. This level uses a mechanism of molecular self-entrapment to immobilize enzymes and cofactors. An enzyme immobilized in self-assembled magnetic nanoparticles is herein referred to as a “bionanocatalyst” (BNC). The invention provides metabolic enzymes such as P450 and supporting enzymes and cofactors incorporated into BNCs. Level 2 is the stabilization of the MNPs into other assemblies such as magnetic or polymeric matrices. In certain embodiments, the BNCs are “templated” onto or into micro or macro structures for commercial or other applications. In one embodiment, the level 2 template is a Magnetic Microparticle (MMP). Level 3 is product conditioning for using the Level 1+2 immobilized enzymes.

In some embodiments, the BNCs of the invention are provided in a magnetic macroporous polymeric hybrid scaffold comprising a cross-linked water-insoluble polymer and an approximately uniform distribution of embedded magnetic microparticles (MMP). The polymer comprises at least polyvinyl alcohol (PVA), has MMPs of about 50-500 nm in size, pores of about 1 to about 50 μm in size, about 20% to 95% w/w MMP, wherein the scaffold comprises an effective surface area for incorporating bionanocatalysts (BNC) that is about total 1-15 m²/g; wherein the total effective surface area for incorporating the enzymes is about 50 to 200 m²/g; wherein the scaffold has a bulk density of between about 0.01 and about 10 g/ml.; and wherein the scaffold has a mass magnetic susceptibility of about 1.0×10⁻³ to about 1×10⁻⁴ m³kg⁻¹. In a preferred embodiment, the magnetic macroporous polymeric hybrid scaffold comprises a contact angle for the scaffold with water that is about 0-90 degrees.

In preferred embodiments, the cross-linked water-insoluble polymer is essentially polyvinyl alcohol (PVA). In more preferred embodiments, the scaffold further comprises a polymer selected from the group consisting of polyethylene, polypropylene, poly-styrene, polyacrylic acid, polyacrylate salt, polymethacrylic acid, polymethacrylate salt, polymethyl methacrylate, polyvinyl acetate, polyvinylfluoride, polyvinylidenefluoride, polytetrafluoroethylene, a phenolic resin, a resorcinol formaldehyde resin, a polyamide, a polyurethane, a polyester, a polyimide, a polybenzimidazole, cellulose, hemicellulose, carboxymethyl cellulose (CMC), 2-hydroxyethylcellulose (HEC), ethylhydroxyethyl cellulose (EHEC), xylan, chitosan, inulin, dextran, agarose, alginic acid, sodium alginate, polylactic acid, polyglycolic acid. a polysiloxane, a polydimethylsiloxane, and a polyphosphazene.

In other more preferred embodiments, the magnetic macroporous polymeric hybrid scaffold comprises PVA and CMC, PVA and alginate, PVA and HEC, or PVA and EHEC. Macroporous polymeric hybrid scaffolds are taught in U.S. Prov. App. No. 62/323,663, incorporated herein by reference in its entirety.

The MNPs allow for a broader range of operating conditions for using enzymes in biocatalytic processes such as temperature, ionic strength, pH, and solvents. The size and magnetization of the MNPs affect the formation and structure of the BNCs. This has a significant impact on the activity of the entrapped enzymes. By virtue of their surprising resilience under various reaction conditions, self-assembled MNP clusters can be used as a superior immobilization material for enzymes that replaces polymeric resins, cross-linked gels, cross-linked enzyme aggregates (CLEAs), cross-linked magnetic beads and the like. Furthermore, they can be used in any application of enzymes on diffusible substrates.

BNC's contain mesopores that are interstitial spaces between the clustered magnetic nanoparticles. Enzymes are immobilized within at least a portion of the mesopores of the magnetic BNCs. As used herein, the term “magnetic” encompasses all types of useful magnetic characteristics, including permanent magnetic, superparamagnetic, paramagnetic, and ferromagnetic behaviors.

BNC sizes of the invention are in the nanoscale, i.e., generally no more than 500 nm. As used herein, the term “size” can refer to a diameter of the magnetic nanoparticle when the magnetic nanoparticle is approximately or substantially spherical. In a case where the magnetic nanoparticle is not approximately or substantially spherical (e.g., substantially ovoid or irregular), the term “size” can refer to either the longest dimension or an average of the three dimensions of the magnetic nanoparticle. The term “size” may also refer to the calculated average size in a population of magnetic nanoparticles.

In different embodiments, the magnetic nanoparticle has a size of precisely, about, up to, or less than, for example, 500 nm, 400 nm, 300 nm, 200 nm, 100 nm, 50 nm, 40 nm, 30 nm, 25 nm, 20 nm, 15 nm, 10 nm, 5 nm, 4 nm, 3 nm, 2 nm, or 1 nm, or a size within a range bounded by any two of the foregoing exemplary sizes.

Within BNCs, the individual magnetic nanoparticles may be primary nanoparticles (i.e., primary crystallites) having any of the sizes provided above. The aggregates of nanoparticles in a BNC are larger in size than the nanoparticles and generally have a size (i.e., secondary size) of at least about 5 nm. In different embodiments, the aggregates have a size of precisely, about, at least, above, up to, or less than, for example, 5 nm, 8 nm, 10 nm, 12 nm, 15 nm, 20 nm, 25 nm, 30 nm, 35 nm, 40 nm, 45 nm, 50 nm, 60 nm, 70 nm, 80 nm, 90 nm, 100 nm, 150 nm, 200 nm, 300 nm, 400 nm, 500 nm, 600 nm, 700 nm, or 800 nm, or a size within a range bounded by any two of the foregoing exemplary sizes.

Typically, the primary and/or aggregated magnetic nanoparticles or BNCs thereof have a distribution of sizes, i.e., they are generally dispersed in size, either narrowly or broadly dispersed. In different embodiments, any range of primary or aggregate sizes can constitute a major or minor proportion of the total range of primary or aggregate sizes. For example, in some embodiments, a particular range of primary particle sizes (for example, at least about 1, 2, 3, 5, or 10 nm and up to about 15, 20, 25, 30, 35, 40, 45, or 50 nm) or a particular range of aggregate particle sizes (for example, at least about 5, 10, 15, or 20 nm and up to about 50, 100, 150, 200, 250, or 300 nm) constitutes at least or above about 50%, 60%, 70%, 80%, 90%, 95%, 98%, 99%, or 100% of the total range of primary particle sizes. In other embodiments, a particular range of primary particle sizes (for example, less than about 1, 2, 3, 5, or 10 nm, or above about 15, 20, 25, 30, 35, 40, 45, or 50 nm) or a particular range of aggregate particle sizes (for example, less than about 20, 10, or 5 nm, or above about 25, 50, 100, 150, 200, 250, or 300 nm) constitutes no more than or less than about 50%, 40%, 30%, 20%, 10%, 5%, 2%, 1%, 0.5%, or 0.1% of the total range of primary particle sizes.

The aggregates of magnetic nanoparticles (i.e., “aggregates”) or BNCs thereof can have any degree of porosity, including a substantial lack of porosity depending upon the quantity of individual primary crystallites they are made of. In particular embodiments, the aggregates are mesoporous by containing interstitial mesopores (i.e., mesopores located between primary magnetic nanoparticles, formed by packing arrangements). The mesopores are generally at least 2 nm and up to 50 nm in size. In different embodiments, the mesopores can have a pore size of precisely or about, for example, 2, 3, 4, 5, 10, 12, 15, 20, 25, 30, 35, 40, 45, or 50 nm, or a pore size within a range bounded by any two of the foregoing exemplary pore sizes. Similar to the case of particle sizes, the mesopores typically have a distribution of sizes, i.e., they are generally dispersed in size, either narrowly or broadly dispersed. In different embodiments, any range of mesopore sizes can constitute a major or minor proportion of the total range of mesopore sizes or of the total pore volume. For example, in some embodiments, a particular range of mesopore sizes (for example, at least about 2, 3, or 5, and up to 8, 10, 15, 20, 25, or 30 nm) constitutes at least or above about 50%, 60%, 70%, 80%, 90%, 95%, 98%, 99%, or 100% of the total range of mesopore sizes or of the total pore volume. In other embodiments, a particular range of mesopore sizes (for example, less than about 2, 3, 4, or 5 nm, or above about 10, 15, 20, 25, 30, 35, 40, 45, or 50 nm) constitutes no more than or less than about 50%, 40%, 30%, 20%, 10%, 5%, 2%, 1%, 0.5%, or 0.1% of the total range of mesopore sizes or of the total pore volume.

The magnetic nanoparticles can have any of the compositions known in the art. In some embodiments, the magnetic nanoparticles are or include a zerovalent metallic portion that is magnetic. Some examples of such zerovalent metals include cobalt, nickel, and iron, and their mixtures and alloys. In other embodiments, the magnetic nanoparticles are or include an oxide of a magnetic metal, such as an oxide of cobalt, nickel, or iron, or a mixture thereof. In some embodiments, the magnetic nanoparticles possess distinct core and surface portions. For example, the magnetic nanoparticles may have a core portion composed of elemental iron, cobalt, or nickel and a surface portion composed of a passivating layer, such as a metal oxide or a noble metal coating, such as a layer of gold, platinum, palladium, or silver. In other embodiments, metal oxide magnetic nanoparticles or aggregates thereof are coated with a layer of a noble metal coating. The noble metal coating may, for example, reduce the number of charges on the magnetic nanoparticle surface, which may beneficially increase dispersibility in solution and better control the size of the BNCs. The noble metal coating protects the magnetic nanoparticles against oxidation, solubilization by leaching or by chelation when chelating organic acids, such as citrate, malonate, or tartrate, are used in the biochemical reactions or processes. The passivating layer can have any suitable thickness, and particularly, at least, up to, or less than, about for example, 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or 10 nm, or a thickness in a range bounded by any two of these values.

Magnetic materials useful for the invention are well-known in the art. Non-limiting examples comprise ferromagnetic and ferromagnetic materials including ores such as iron ore (magnetite or lodestone), cobalt, and nickel. In other embodiments, rare earth magnets are used. Non-limiting examples include neodymium, gadolinium, sysprosium, samarium-cobalt, neodymium-iron-boron, and the like. In yet further embodiments, the magnets comprise composite materials. Non-limiting examples include ceramic, ferrite, and alnico magnets. In preferred embodiments, the magnetic nanoparticles have an iron oxide composition. The iron oxide composition can be any of the magnetic or superparamagnetic iron oxide compositions known in the art, e.g., magnetite (FesO/O, hematite (α-Fe2θ 3), maghemite (γ-Fe2C>3), or a spinel ferrite according to the formula AB₂O₄, wherein A is a divalent metal (e.g., Xn²⁺, Ni²⁺, Mn²⁺, Co²⁺, Ba²⁺, Sr²⁺, or combination thereof) and B is a trivalent metal (e.g., Fe³⁺, Cr³⁺, or combination thereof).

In some embodiments, the BNC's are formed by exploiting the instability of superparamagnetic NPs. The Point of Zero Charges (PZC) of magnetite is pH 7.9, around which magnetic NPs cannot repel each other and cluster readily. NPs are positively charged below the PZC and negatively charged above it. Cluster formation may be driven by electrostatic Interactions. The opposite electrostatic charges at the surface of the enzymes from charged amino acids can compensate the surface charge of the NPs. Enzymes can be assimilated to poly-anions or poly-cations that neutralize the charge of multiple NPs. Each enzyme has its own isoelectric point (pI) and surface composition of charged amino acids that will trigger the aggregation of nanoparticles. The enzymes may then be entrapped and stabilized in mesoporous clusters. Initial NP and enzyme concentrations, pH and ionic strength are the main parameters controlling the aggregation rate and final cluster size. The size of the clusters greatly influences the efficacy of the reaction because of mass transport limitations of the substrates and products in-and-out of the clusters. They can be tuned from 100 nm to 10 μm clusters to control the enzyme loading and the substrate diffusion rates.

Entrapped enzymes are referred to Level 1. “Locked” clusters in rigid scaffolds may result from templating them onto or within bigger or more stable magnetic or polymeric scaffolds, referred as Level 2. This prevents over-aggregation and adds mass magnetization for ease of capture by external magnets.

In particular embodiments, the above mesoporous aggregates of magnetic nanoparticles (BNCs) are incorporated into a continuous macroporous scaffold to form a hierarchical catalyst assembly with first and second levels of assembly. The first level of assembly is found in the BNCs. The second level of assembly is found in the incorporation of the BNCs into the continuous macroporous scaffold. In some embodiments, the level 2 assembly is magnetic.

The term “continuous” as used herein for the macroporous magnetic scaffold, indicates a material that is not a particulate assembly, i.e., is not constructed of particles or discrete objects assembled with each other to form a macroscopic structure. In contrast to a particulate assembly, the continuous structure is substantially seamless and uniform around macropores that periodically interrupt the seamless and uniform structure. The macropores in the continuous scaffold are, thus, not interstitial spaces between agglomerated particles. Nevertheless, the continuous scaffold can be constructed of an assembly or aggregation of smaller primary continuous scaffolds, as long as the assembly or aggregation of primary continuous scaffolds does not include macropores (e.g., greater than about 50 nm and up to about 100) formed by interstitial spaces between primary continuous scaffolds. Particularly in the case of inorganic materials such as ceramics or elemental materials, the continuous scaffold may or may not also include crystalline domains or phase boundaries.

In particular embodiments, the above mesoporous aggregates of magnetic nanoparticles (BNCs) are incorporated into a continuous macroporous scaffold to form a hierarchical catalyst assembly with first and second levels of assembly. The first level of assembly is found in the BNCs. The second level of assembly is found in the incorporation of the BNCs into the continuous macroporous scaffold. The overall hierarchical catalyst assembly is magnetic by at least the presence of the BNCs.

The macroporous scaffold contains macropores (i.e., pores of a macroscale size) having a size greater than 50 nm. In different embodiments, the macropores have a size of precisely, about, at least, above, up to, or less than, for example, 60 nm, 70 nm, 80 nm, 90 nm, 100 nm, 150 nm, 200 nm, 300 nm, 400 nm, 500 nm, 600 nm, 700 nm, 800 nm, 900 nm, 1 micron (1 μm), 1.2 μm, 1.5 μm, 2 μm, 3 μm, 4 μm, 5 μm, 10 μm, 20 μm, 30 μm, 40 μm, 50 μm, 60 μm, 70 μm, 80 μm, 90 μm, or 100 μm, or a size within a range bounded by any two of the foregoing exemplary sizes.

The macroporous scaffold can have any suitable size as long as it can accommodate macropores. In typical embodiments, the macroporous scaffold possesses at least one size dimension in the macroscale. The at least one macroscale dimension is above 50 nm, and can be any of the values provided above for the macropores, and in particular, a dimension of precisely, about, at least, above, up to, or less than, for example, 1 μm, 2 μm, 3 μm, 4 μm, 5 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 82 m, 1 mm, 2 mm, 5 mm, or 1 cm, or a size within a range bounded by any two of the foregoing exemplary sizes. Where only one or two of the size dimensions are in the macroscale, the remaining one or two dimensions can be in the nanoscale, such as any of the values provided above for the magnetic nanoparticles (e.g., independently, precisely, about, at least, above, up to, or less than, for example, 1, 2, 3, 4, 5, 10, 20, 30, 40, 50, 60, 70, 80, 90, or 100 nm, or a value within a range bounded by any two of the foregoing values). In some embodiments, at least two or all of the size dimensions of the macroporous scaffold is in the macroscale.

In a first set of embodiments, the continuous macroporous scaffold in which the BNCs are incorporated is magnetic, i.e., even in the absence of the BNCs. The continuous macroporous scaffold can be magnetic by, for example, being composed of a magnetic polymer composition. An example of a magnetic polymer is PANiCNQ, which is a combination of tetracyanoquinodimethane (TCNQ) and the emeraldine-based form of polyaniline (PANi), as well known in the art. Alternatively, or in addition, the continuous macroporous scaffold can be magnetic by having embedded therein magnetic particles not belonging to the BNCs. The magnetic particles not belonging to the BNCs may be, for example, magnetic nano- or micro-particles not associated with an FRP enzyme or any enzyme. The magnetic microparticles may have a size or size distribution as provided above for the macropores, although independent of the macropore sizes. In particular embodiments, the magnetic microparticles have a size of about, precisely, or at least 20, 30, 40, 50, 60, 70, 80, 90, 100, 100, 200, 300, 400, 500, 600, 700, 800, 900, 1000 nm, or a size within a range bounded by any two of the foregoing exemplary sizes. In some embodiments, the continuous macroporous scaffold has embedded therein magnetic microparticles that are adsorbed to at least a portion of the BNCs, or wherein the magnetic microparticles are not associated with or adsorbed to the BNCs.

In a second set of embodiments, the continuous scaffold in which the BNCs are incorporated is non-magnetic. Nevertheless, the overall hierarchical catalyst assembly containing the non-magnetic scaffold remains magnetic by at least the presence of the BNCs incorporated therein.

In one embodiment, the continuous macroporous scaffold (or precursor thereof) has a polymeric composition. The polymeric composition can be any of the solid organic, inorganic, or hybrid organic-inorganic polymer compositions known in the art, and may be synthetic or a biopolymer that acts as a binder. Preferably, the polymeric macroporous scaffold does not dissolve or degrade in water or other medium in which the hierarchical catalyst is intended to be used. Some examples of synthetic organic polymers include the vinyl addition polymers (e.g., polyethylene, polypropylene, polystyrene, polyacrylic acid or polyacrylate salt, polymethacrylic acid or polymethacrylate salt, poly(methylmethacrylate), polyvinyl acetate, polyvinyl alcohol, and the like), fluoropolymers (e.g., polyvinylfluoride, polyvinylidenefluoride, polytetrafluoroethylene, and the like), the epoxides (e.g., phenolic resins, resorcinol-formaldehyde resins), the polyamides, the polyurethanes, the polyesters, the polyimides, the polybenzimidazoles, and copolymers thereof. Some examples of biopolymers include the polysaccharides (e.g., cellulose, hemicellulose, xylan, chitosan, inulin, dextran, agarose, and alginic acid), polylactic acid, and polyglycolic acid. In the particular case of cellulose, the cellulose may be microbial- or algae-derived cellulose. Some examples of inorganic or hybrid organic-inorganic polymers include the polysiloxanes (e.g., as prepared by sol gel synthesis, such as polydimethylsiloxane) and polyphosphazenes. In some embodiments, any one or more classes or specific types of polymer compositions provided above are excluded as macroporous scaffolds.

In another embodiment, the continuous macroporous scaffold (or precursor thereof) has a non-polymeric composition. The non-polymeric composition can have, for example, a ceramic or elemental composition. The ceramic composition may be crystalline, polycrystalline, or amorphous, and may have any of the compositions known in the art, including oxide compositions (e.g., alumina, beryllia, ceria, yttria, or zirconia) and non-oxide compositions (e.g., carbide, silicide, nitride, boride, or sulfide compositions). The elemental composition may also be crystalline, polycrystalline, or amorphous, and may have any suitable elemental composition, such as carbon, aluminum, or silicon.

In other embodiments, the BNCs reside in a non-continuous macroporous support containing (or constructed of) an assembly (i.e., aggregation) of Magnetic Microparticles (MMPs) that includes macropores as interstitial spaces between the magnetic microparticles. The magnetic microparticles are typically ferromagnetic and can be made of magnetite or other ferromagnetic materials. The BNCs are embedded in at least a portion of the macropores of the aggregation of magnetic microparticles, and may also reside on the surface of the magnetic microparticles. The BNCs can associate with the surface of the magnetic microparticles by magnetic interaction. The magnetic microparticles may or may not be coated with a metal oxide or noble metal coating layer. In some embodiments, the BNC-MMP assembly is incorporated (i.e., embedded) into a continuous macroporous scaffold, as described above, to provide a hierarchical catalyst assembly.

In some embodiments, the scaffolds comprise cross-linked water-insoluble polymers and an approximately uniform distribution of embedded magnetic microparticles (MMP). The cross-linked polymer comprises polyvinyl alcohol (PVA) and optionally additional polymeric materials. The scaffolds may take any shape by using a cast during preparation of the scaffolds. Alternatively, the scaffolds may be ground to microparticles for use in biocatalyst reactions. Alternatively, the scaffolds may be shaped as beads for use in biocatalyst reactions. Alternatively, the scaffolds may be monoliths. Methods for preparing and using the scaffolds are also provided.

In other embodiments, the magnetic macroporous polymeric hybrid scaffold comprises a cross-linked water-insoluble polymer and an approximately uniform distribution of embedded magnetic microparticles (MMP). The polymer comprises at least polyvinyl alcohol (PVA), has MMPs of about 50-500 nm in size, pores of about 1 to about 50 μm in size, about 20% to 95% w/w MMP, wherein the scaffold comprises an effective surface area for incorporating bionanocatalysts (BNC) that is about total 1-15 m²/g; wherein the total effective surface area for incorporating the enzymes is about 50 to 200 m²/g; wherein the scaffold has a bulk density of between about 0.01 and about 10 g/ml.; and wherein the scaffold has a mass magnetic susceptibility of about 1.0×10⁻³ to about 1×10⁻⁴ m³kg⁻¹. In a preferred embodiment, the magnetic macroporous polymeric hybrid scaffold comprises a contact angle for the scaffold with water that is about 0-90 degrees. Details of the macroporous polymeric hybrid scaffold embodiments are taught in U.S. Provisional App. No. 62/323,663, incorporated herein by reference in its entirety.

The individual magnetic nanoparticles or aggregates thereof or BNCs thereof possess any suitable degree of magnetism. For example, the magnetic nanoparticles, BNCs, or BNC scaffold assemblies can possess a saturated magnetization (Ms) of at least or up to about 5, 10, 15, 20, 25, 30, 40, 45, 50, 60, 70, 80, 90, or 100 emu/g. The magnetic nanoparticles, BNCs, or BNC-scaffold assemblies preferably possess a remanent magnetization (Mr) of no more than (i.e., up to) or less than 5 emu/g, and more preferably, up to or less than 4 emu/g, 3 emu/g, 2 emu/g, 1 emu/g, 0.5 emu/g, or 0.1 emu/g. The surface magnetic field of the magnetic nanoparticles, BNCs, or BNC-scaffold assemblies can be about or at least, for example, about 0.5, 1, 5, 10, 50, 100, 200, 300, 400, 500, 600, 700, 800, 900, or 1000 Gauss (G), or a magnetic field within a range bounded by any two of the foregoing values. If microparticles are included, the microparticles may also possess any of the above magnetic strengths.

The magnetic nanoparticles or aggregates thereof can be made to adsorb a suitable amount of enzyme, up to or below a saturation level, depending on the application, to produce the resulting BNC. In different embodiments, the magnetic nanoparticles or aggregates thereof may adsorb about, at least, up to, or less than, for example, 1, 5, 10, 15, 20, 25, or 30 pmol/m2 of enzyme. Alternatively, the magnetic nanoparticles or aggregates thereof may adsorb an amount of enzyme that is about, at least, up to, or less than, for example, about 10%, 20%, 30%, 40%, 50%, 60%, 70%, 80%, 90%, or 100% of a saturation level.

The magnetic nanoparticles or aggregates thereof or BNCs thereof possess any suitable pore volume. For example, the magnetic nanoparticles or aggregates thereof can possess a pore volume of about, at least, up to, or less than, for example, about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 cm3/g, or a pore volume within a range bounded by any two of the foregoing values.

The magnetic nanoparticles or aggregates thereof or BNCs thereof possess any suitable specific surface area. For example, the magnetic nanoparticles or aggregates thereof can have a specific surface area of about, at least, up to, or less than, for example, about 50, 60, 70, 80, 90, 100, 110, 120, 130, 140, 150, 160, 170, 180, 190, or 200 m 2/g.

MNPs, their structures, organizations, suitable enzymes, and uses are described in WO2012122437, WO2014055853, Int'l Application No. PCT/US16/31419, and U.S. Provisional Application Nos. 62/193,041 and 62/323,663, incorporated by reference herein in their entirety.

Automated continuous production of BNCs are disclosed in U.S. Provisional Application No. 62/193,041, incorporated by reference herein in its entirety.

The invention provides BNCs having magnetically-entrapped monooxygenases (E.C.1.13). In one embodiment, the monooxygenase is P450 (EC_1.14.-.-)). In a preferred embodiment, the monoxygenase is of human origin. (See, e.g., https://www.ncbi.nlm.nih.gov/pmc/articles/PMC2884625/.) In another preferred embodiment, the monoxygenase is of bacterial origin. In other preferred embodiments, the monoxygenase is of algal, fungal, plant or animal origin.

In some embodiments, the P450 is in a soluble form such as the BM3 P450 from Bacillus megaterium. See, e.g., SEQ ID NO:1. In other embodiments, the BM3 P450 has one or more variant amino acids from the wild-type. In other embodiments, the P450 has at least a 90% sequence identity to SEQ ID NO:1.

In some embodiments, the P450 is Human. In other embodiments, the human P450 is in an insoluble form and is embedded in the membranes of small vesicular organelles. The organelles may contain other enzymes that work with or enhance the activity of the monooxygenases. In other embodiments, the P450 is in a supersome. (See, e.g., Corning, https://www.corning.com/worldwide/en/products/life-sciences/products/adme-tox-research/recombinant-metabolic-enzymes.html.) In other embodiments, the P450 is in a bactosome. (See, e.g., Cypex, http://www.cypex.co.uk/ezcypbuf.htm.)

In some embodiments, the P450 monooxygenase comprises a P450 sequence that is of an origin selected from the group consisting of primate, mouse, rat, dog, cat, horse, cow, sheep, and goat, or derivatives thereof. In other embodiments, the P450 monooxygenase comprises a P450 sequence that is of an origin selected from the group consisting of insect, fish, fungus, yeast, protozoan, and plant.

Cytochrome p450s (CYPs) (EC 1.14.13.-) are a diverse family of NAPDH-dependent oxidative hemeproteins present in all organisms. These enzymes, with expression profiles differing between tissues, carry out the metabolism of xenobiotics, or non-endogenous chemicals. (Denisov et al., Chem. Rev. 105(6):2253-78 (2005), incorporated by reference herein in its entirety.) CYPs generate metabolites with higher solubility than their parent compounds to facilitate clearance from the body. The substrate range of CYPs is broad and varies between isoforms, which are capable of performing hydroxylation, epoxidation, deamination, dealkylation, and dearylation reactions, among others.

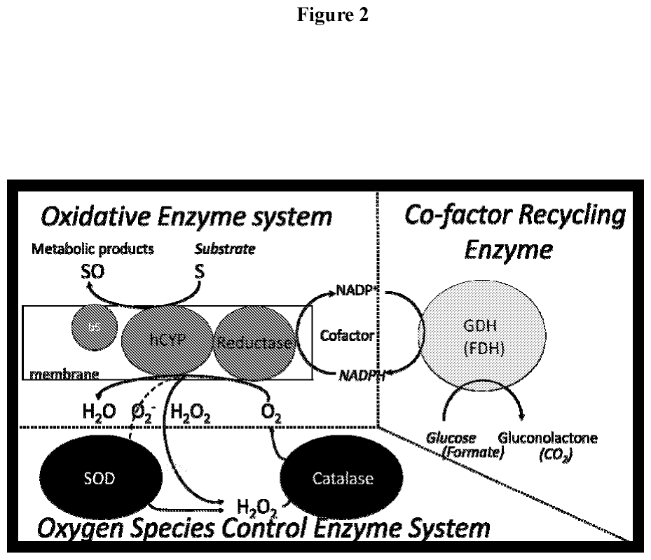

As part of safety due diligence for drugs, consumer products, and food additive development, tissue microsomes and recombinant CYPs are used to generate metabolites for evaluation of their toxicity. However, CYPs are notoriously challenging to use in industry as they often have low process stability and succumb to oxidative denaturation because of reactive oxygen species (ROS) formed as side products of CYP-mediated oxidations. Human CYPs are membrane bound and localize in the endoplasmic reticulum near cytochrome P450 reductase (CPR) and cytochrome b5, the latter sometimes improving CYP activity and the former required for activity. (FIG. 2.)

The P450s of the invention may perform aliphatic hydroxylations, aromatic hydroxylations, epoxidations, heteroatom dealkylation, alkyne oxygenations, heteroatom oxygenations, aromatic epoxidations and NIH-shift, dehalogenations, dehydrogenations, reduction and cleavage of esters.

The invention provides using other metabolic enzymes in the BNCs that produce metabolites in Phase I, II and III metabolism. Examples include UDP-glucuronosyl transferases, sulfotransferases, flavin-containing monooxygenases, monoamine oxidases, and carboxyesterases.

UDP-glucuronosyl transferases (UGT, EC2.4.1.17) enzymes catalyze the addition of a glucuronic acid moiety to xenobiotics. UGT's pathway is a major route of the human body's elimination of frequently prescribed drugs, xenobiotics, dietary substances, toxins, and endogenous toxins.

The superfamily of Sulfotransferases (E.C. 2.8.2.) are transferase enzymes that catalyze the transfer of a sulfo group from a donor molecule to an acceptor alcohol or amine. The most common sulfo group donor is 3′-phosphoadenosine-5′-phosphosulfate (PAPS). In the case of most xenobiotics and small endogenous substrates, sulfonation has generally been considered a detoxification pathway leading to more water-soluble products and thereby aiding their excretion via the kidneys or bile.

The flavin-containing monooxygenase (FMO, E.C. 1.14.13.8) enzymes perform the oxidation of xenobiotics to facilitate their excretion. These enzymes can oxidize a wide array of heteroatoms, particularly soft nucleophiles, such as amines, sulfides, and phosphites. This reaction requires dioxygen, an NADPH cofactor, and an FAD prosthetic group.

Monoamine oxidases (MAO, E.C. 1.4.3.4) catalyze the oxidative deamination of monoamines. Oxygen is used to remove an amine group from a molecule, resulting in the corresponding aldehyde and ammonia. MAO are well known enzymes in pharmacology, since they are the substrate for the action of a number of monoamine oxidase inhibitor drugs.

Carboxylesterases (E.C. 3.1.1.1) convert carboxylic esters and H₂O to alcohol and carboxylate. They are common in mammalian livers and participate in the metabolism of xenobiotics such as toxins or drugs; the resulting carboxylates are then conjugated by other enzymes to increase solubility and are eventually eliminated.

In some embodiments, the oxidoreductase of the invention is a catalase. Catalases (EC. 1.11.1.6) are enzymes found in nearly all living organisms exposed to oxygen. They catalyze the decomposition of hydrogen peroxide (H₂O₂) to water and oxygen (O₂). They protect cells from oxidative damage by reactive oxygen species (ROS). Catalases have some of the highest turnover numbers of all enzymes; typically one catalase molecule can convert millions of hydrogen peroxide molecules to water and oxygen each second. Catalases are tetramers of four polypeptide chains, each over 500 amino acids long. They contain four porphyrin heme (iron) groups that allow them to react with the hydrogen peroxide. Catalases are used in the food industry, e.g., for removing hydrogen peroxide from milk prior to cheese production and for producing acidity regulators such as gluconic acid. Catalases are also used in the textile industry for removing hydrogen peroxide from fabrics.

In other embodiments, the oxidoreductase of the invention is a superoxide dismutase (e.g., EC 1.15.1.1). These are enzymes that alternately catalyzes the dismutation of the superoxide (O₂-) radical into either ordinary molecular oxygen (O₂) or hydrogen peroxide (H₂O₂). Superoxide is produced as a by-product of oxygen metabolism and, if not regulated, causes oxidative damage. Hydrogen peroxide is also damaging but can be degraded by other enzymes such as catalase.

In other embodiments, the oxidoreductase is a glucose oxidase (e.g. Notatin, EC 1.1.3.4). It catalyzes the oxidation of glucose to hydrogen peroxide and D-glucono-δ-lactone. It is used, for example, to generate hydrogen peroxide as an oxidizing agent for hydrogen peroxide consuming enzymes such as peroxidase.

In other embodiments, the metabolic enzyme is a carboxylesterase (EC 3.1.1.1). Carboxylesterases are widely distributed in nature, and are common in mammalian liver. Many participate in phase I metabolism of xenobiotics such as toxins or drugs; the resulting carboxylates are then conjugated by other enzymes to increase solubility and eventually excreted. The carboxylesterase family of evolutionarily related proteins (those with clear sequence homology to each other) includes a number of proteins with different substrate specificities, such as acetylcholinesterases.

The invention provides magnetically immobilized P450 catalytic systems for the production of chemical metabolites of P450. In some embodiments, enzyme stability or activity is maximized while reducing cofactor requirements. In other embodiments, the enzymes are immobilized on reusable magnetic carriers for metabolite manufacturing. In other embodiments, the magnetically immobilized P450 increases chemical manufacturing production capacity, enhances enzyme recovery, or decreases costs and environmental pollution. In other embodiments of the invention there is minimal to no loss in enzyme activity. In preferred embodiments, only about 1, 2, 3, 4, 5, 6, 7, 8, 9, 10, 11, 12, 13, 14, 15, 16-20, or 20-30% of the enzyme activity is lost. In other embodiments of the invention, there is an increase in enzyme activity and productivity. In other embodiments, one or more enzymes in addition to P450 are magnetically immobilized. This may facilitate the adoption of magnetic materials coupled with magnetic processes into existing manufacturing infrastructures or enable green chemistry methods.

The invention provides P450 metabolic enzymes/BNC-based biocatalytic syntheses that produce biologically relevant metabolites that are otherwise difficult to synthesize by traditional chemistry. In some embodiments, the invention mimics the diversity of metabolites that are produced by organisms upon exposure to xenobiotics. This is particularly relevant in the evaluation of drugs where oxidized metabolites can have adverse effects, or on the contrary, have higher pharmacological effects than a parent molecule from which it is derived. Here, metabolic profiling may increase the safety of new drugs. (See Metabolites in Safety Testing guideline by the U.S. Food and Drug Administration (FDA), http://www.fda.gov/downloads/Drugs/.../Guidances/ucm079266.pdf, incorporated by reference herein in its entirety.) Metabolic profiling of drugs and chemicals, in general, is limited by the difficulty of producing sufficient quantities of biologically relevant metabolites or by the difficulty of producing a diversity of metabolites in a high-throughput fashion.

The P450 cytochromes represent a gene superfamily of enzymes that are responsible for the oxidative metabolism of a wide variety of xenobiotics, including drugs. Wrighton and Stevens, Crit. Rev. Tox. 22(1):1-21 (1992); Kim et al., Xenobiotica 27(7):657-665 (1997): Tang, et al. J. Pharm. Exp. Therap., 293(2):453-459 (2000); Zhu et al., Drug Metabolism and Disposition 33(4):500-507 (2005); Trefzer et al. Appl. Environ. Microbiol. 73(13):4317-4325 (2007); Dresser et al. Clinical Pharmacokinetics 38(1):41-57 (2012). To generate drug metabolites in drug development, human liver microsomes, human-recombinant microsomes, or purified human-recombinant P450 monooxygenases are commercially available but typically suffer from process instability and poor activity levels. Iribarne, et al., Chem. Res. Tox. 9(2): p. 365-373 (1996); Yamazaki et al., Chem. Res. Tox. 11(6): p. 659-665 (1998); Joo et al., Nature, 399(6737):670-673 (1999). The foregoing are incorporated by reference in their entirety.

The P450 BNCs of the invention may be used, for example, in drug or specialty chemical manufacturing. In some embodiments, the manufactured compounds are small molecules. In other embodiments, the manufactured compounds are active pharmaceutical ingredients (API). In other embodiments, the manufactured compounds are active agricultural ingredients such as pesticides. In other embodiments, the manufactured compounds are active ingredients such as hormones and pheromones. In other embodiments, the manufactured compounds are flavors, fragrances and food coloring.

P450 enzymes are labile and notoriously difficult to use in biocatalytic reactions. They are, however, a major component of the metabolic pathway of drug and xenobiotic conversions and hence play a major role in the generation of drug metabolites. Human P450 have a broad range of substrates. For example, human CYP1A1 converts EROD to resofurin; human CYP1A2 converts phenacetin to acetaminophen and is also active on Clozapine, Olanzepine, Imipramine, Propranolol, and Theophylline; human CYP2A6 converts coumarin to 7-hydroxycoumarin; human CYP2B6 converts bupropion to hydroxybupropion and is also active Cyclophosphamide, Efavirenz, Nevirapine, Artemisisin, Methadone, and Profofol; human CYP2C8 converts Paclitaxel to 6α-hydroxypaclitaxel; human CYP2C9 converts diclofenac to 4′-hydroxydiclofenac and is also active Flurbiprofen, Ibuprofen, Naproxen, Phenytoin, Piroxicam Tolbutamide and Warfarin; human CYP2C19 converts mephenytoin to 4′-hydroxyphenytoin and is also active Amitriptyline, Cyclophosphamide, Diazepam, Imipramine, Omeprazole, and Phenytoin; human CYP2D6 converts dextromethorphan to dextrorphan and also also active on Amitriptyline, Imipramine, Propranolol, Codeine, Dextromethorphan, Desipramine and Bufaralol; human CYP2E1 is active on chlorzoxazone to 6-hydroxychlorzoxazone and also coverts Acetaminophen; human CYP2A4 converts midazolam to 1-hydroxymidazolam and is also active Alprazolam, Carbamazepine, Testerone, Cyclosporine, Midazolam, Simvastatin, Triazolam and Diazepam.

Other metabolic enzymes such as human UGT, convert, for example, 7-hydroxycoumarin to 7-hydroxycoumarin glucuronide and human SULT converts 7-hydroxycoumarin to 7-hydroxycoumarin sulftate.

One difficulty in using monooxygenases in industrial processes is cofactor regeneration, and in particular, β-1,4-nicotinamide adenine dinucleotide phosphate (NADPH). NADPH is too expensive to be used stoichiometrically. Thus, in some embodiments, the invention provides cofactor regeneration compositions and methods to be used with the P450 BNCs. In preferred embodiments, the BNCs are used along with recycling enzymes. In more preferred embodiments, the recycling enzyme is Glucose Dehydrogenase (GDH). In other preferred embodiments, recycling enzymes such as GDH are co-immobilized with a P450.

The invention provides a process for the use of P450 metabolic enzymes magnetically-immobilized into BNCs. In some embodiments, machines provide magnetic mixing and capture P450.

The invention provides enzymes that are expressed from a nucleic acid encoding enzyme polypeptides. In certain embodiments, the recombinant nucleic acids encoding an enzyme polypeptide may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells.

Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding an enzyme polypeptide operably linked to at least one regulatory sequence. Regulatory sequence for use herein include promoters, enhancers, and other expression control elements. In certain embodiments, an expression vector is designed considering the choice of the host cell to be transformed, the particular enzyme polypeptide desired to be expressed, the vector's copy number, the ability to control that copy number, or the expression of any other protein encoded by the vector, such as antibiotic markers.

Another aspect includes screening gene products of combinatorial libraries generated by the combinatorial mutagenesis of a nucleic acid described herein. Such screening methods include, for example, cloning the gene library into replicable expression vectors, transforming appropriate cells with the resulting library of vectors, and expressing the combinatorial genes under conditions to form such library. The screening methods optionally further comprise detecting a desired activity and isolating a product detected. Each of the illustrative assays described below are amenable to high-throughput analysis as necessary to screen large numbers of degenerate sequences created by combinatorial mutagenesis techniques.

Certain embodiments include expressing a nucleic acid in microorganisms. One embodiment includes expressing a nucleic acid in a bacterial system, for example, in Bacillus brevis, Bacillus megaterium, Bacillus subtilis, Caulobacter crescentus, Escherichia coli and their derivatives. Exemplary promoters include the 1-arabinose inducible araBAD promoter (PBAD), the lac promoter, the 1-rhamnose inducible rhaP BAD promoter, the T7 RNA polymerase promoter, the trc and tac promoter, the lambda phage promoter Pl, and the anhydrotetracycline-inducible tetA promoter/operator.

Other embodiments include expressing a nucleic acid in a yeast expression system. Exemplary promoters used in yeast vectors include the promoters for 3-phosphoglycerate kinase (Hitzeman et al., J. Biol. Chem. 255:2073 (1980)); other glycolytic enzymes (Hess et al., J. Adv. Enzyme Res. 7:149 (1968); Holland et al., Biochemistry 17:4900 (1978). Others promoters are from, e.g., enolase, glyceraldehyde-3-phosphate dehydrogenase, hexokinase, pyvurate decarboxylase, phosphofructokinase, glucose-6-phosphate isomerase, 3-phosphoglycerate mutase, pyruvate kinase, triosephosphate somerase, phosphoglucose isomerase, glucokinase alcohol oxidase I (AOX1), alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter and termination sequences, with or without an origin of replication, is suitable. Certain yeast expression systems are commercially available, for example, from Clontech Laboratories, Inc. (Palo Alto, Calif , e.g. Pyex 4T family of vectors for S. cerevisiae), Invitrogen (Carlsbad, Calif., e.g. Ppicz series Easy Select Pichia Expression Kit) and Stratagene (La Jolla, Calif., e.g. ESP.TM Yeast Protein Expression and Purification System for S. pombe and Pesc vectors for S. cerevisiae).

Other embodiments include expressing a nucleic acid in mammalian expression systems. Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In a specific embodiment, a yeast alcohol oxidase promoter is used.

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

Other embodiments include expressing a nucleic acid in insect cell expression systems. Eukaryotic expression systems employing insect cell hosts may rely on either plasmid or baculoviral expression systems. Typical insect host cells are derived from the fall army worm (Spodoptera frugiperda). For expression of a foreign protein these cells are infected with a recombinant form of the baculovirus Autographa californica nuclear polyhedrosis virus which has the gene of interest expressed under the control of the viral polyhedron promoter. Other insects infected by this virus include a cell line known commercially as “High 5” (Invitrogen) which is derived from the cabbage looper (Trichoplusia ni). Another baculovirus sometimes used is the Bombyx mori nuclear polyhedorsis virus which infect the silk worm (Bombyx mori). Numerous baculovirus expression systems are commercially available, for example, from Thermo Fisher (Bac-N-Blue™k or BAC-TO-BAC™ Systems), Clontech (BacPAK™ Baculovirus Expression System), Novagen (Bac Vector System™), or others from Pharmingen or Quantum Biotechnologies. Another insect cell host is the common fruit fly, Drosophila melanogaster, for which a transient or stable plasmid based transfection kit is offered commercially by Thermo Fisher (The DES™ System).

In some embodiments, cells are transformed with vectors that express a nucleic acid described herein. Transformation techniques for inserting new genetic material into eukaryotic cells, including animal and plant cells, are well known. Viral vectors may be used for inserting expression cassettes into host cell genomes. Alternatively, the vectors may be transfected into the host cells. Transfection may be accomplished by calcium phosphate precipitation, electroporation, optical transfection, protoplast fusion, impalefection, and hydrodynamic delivery.

Certain embodiments include expressing a nucleic acid encoding an enzyme polypeptide in in mammalian cell lines, for example Chinese hamster ovary cells (CHO) and Vero cells. The method optionally further comprises recovering the enzyme polypeptide.

In some embodiments, the enzymes of the invention are homologous to naturally-occurring enzymes. “Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least a 50, 55, 60, 65, 70, 75, 85, 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

Another aspect of the invention includes enzyme polypeptides that are synthesized in an in vitro synthesis reaction. In an example, the in vitro synthesis reaction is selected from the group consisting of cell-free protein synthesis, liquid phase protein synthesis, and solid phase protein synthesis as is well-known in the art.

In order that the invention described herein may be more fully understood, the following examples are set forth. It should be understood that these examples are for illustrative purposes only and are not to be construed as limiting this invention in any manner.

### EXAMPLES

### Example 1

### Co-Immobilization of Bacterial BM3p450 Cytochrome with Glucose Dehydrogenase, Catalase, Superoxide Dismutase, and NADPH into Magnetic Supports

Bacterial P450 BM3 (also known as CYP102A1) derived from Bacillus megaterium, P450 was used in this example because it can be expressed at high levels in (˜12% dry cell mass), and, unlike nearly all other CYPs, its hydroxylase, reductase and electron-transfer domains are all in one contiguous polypeptide chain. (Sawayama et al., Chemistry 15(43):11723-9 (2009), incorporated herein by reference in its entirety.) A magnetically-immobilized BM3 fusion protein (MW≈120 kDa) showed efficient and recyclable fatty-acid hydroxylase activity. The final loading was targeted to be around 80% (g/g) of BM3 in the BNCs then templated onto ground magnetic macroporous polymeric hybrid scaffolds for a 1% total protein loading. The immobilization yield in the BNCs was 100%. The purity of the crude extract was around 30% content of BM3. This resulted in BMCs with 0.3% CYP loading. NADPH was co-immobilized along with GDH for cofactor recycling. SOD and CAT were also co-immobilized for the control of ROS.

Materials and Equipment. Recombinant BM3 Cytochrome P450 active on p-nitrophenyl laurate expressed in Bacillus megaterium and a bacterial glucose dehydrogenase (GDH) expressed in E. coli was used. Bovine serum albumin (BSA), Bovine liver catalase (CAT), Bovine erythrocyte cytosolic superoxide dismutase (SOD) expressed in E. coli, glucose (beta-d-glucose), p-nitrophenyl laurate (p-NPL), p-nitrophenol (p-NP), nicotinamide adenine dinucleotide phosphate (reduced) tetrasodium salt (NADPH), were purchased from Sigma-Aldrich (St. Louis, Mo., USA). Dimethyl sulfoxide (DMSO) was purchased from Fisher Scientific (Fair Lawn, N.J., USA). Hydrochloric acid, sodium hydroxide, magnesium chloride, and phosphate buffer salts were from Macron Fine Chemicals (Center Valley, Pa., USA). The Quick Start™ Bradford Protein Assay was purchased from Bio-Rad (Hercules, Calif., USA). Stock solutions were made with 18.2 MΩ-cm water purified by Barnstead™ Nanopure™. Absorbance was measured in triplicate in Costar™ 3635 UV-transparent microplates using a Biotek Synergy4™ plate reader operated with Gen5™ software. A sonicator (FB-505) with a ⅛″ probe was purchased from Fisher Scientific® (Waltham, Mass.). ZymTrap™, (powder, 100-500 μm, MO32-40, Zymtronix, Ithaca N.Y., Corgié et al., Chemistry Today, 34:15-20 (2016), incorporated by reference herein in its entirety) was used as a magnetic scaffold for the immobilized P450 enzyme systems.

Reagents. BM3 was obtained from lyophilized crude extracts of bacteria in which it was recombinantly expressed. All aqueous stocks were prepared with ultrapure (MQ) water. Lyophilized BM3, GDH, and NADPH were dissolved in ice-cold oxygen free 2 mM PBS, pH 7.4 and prepared fresh daily. CYP and GDH were centrifuged at 4° C. at 12000 g for 10 min to pellet cell debris. Their supernatants were collected and protein content quantified using the Bradford assay with BSA standards. p-NPL and p-NP stock solutions were prepared in pure DMSO to 100 mM and stored at 4° C. Magnesium chloride (1M) and glucose (100 mM) were dissolved in water and stored at 4° C. All stock solutions were kept on ice. Dilutions were made just before use in assays and allowed to equilibrate to room temperature (21° C.).

Immobilization. BM3 immobilizations were optimized using the methods taught in Int'l Pub. Nos. WO2012122437 and WO2014055853, U.S. Prov. App. No. 62/323,663, and Corgié et al., Chemistry Today, 34:15-20 (2016). The foregoing are incorporated by reference herein in their entirety. Immobilized, non-CYP biological and chemical components were referred to as the CYP Support System (SS): GDH for cofactor regeneration, CAT/SOD for reactive oxygen species (ROS) control, and NADPH for stability during immobilization. Free CYP/GDH/CAT/SOD/NADPH stock (500 μg/mL CYP, 100:100:1:1:100 molar ratios) was prepared in cold buffer using fresh enzyme stocks. A 5 mL 2500 μg/ml MNP stock was sonicated at a 40% amplitude for 1 min, equilibrated to room temperature using a water bath, and its pH was adjusted to 3. Free CYP+SS (500 μL) and an equal volume of sonicated MNPs was dispensed into a 2 mL microcentrifuge tube then pipette mixed 10 times. CYP+SS BMCs were prepared by adding 1 mL of BNCs to 48.75 mg MO32-40 ZymTrap powder and 10 times. These BMCs were gently mixed on a rotator for 1 h then pelleted magnetically. Their supernatants were saved for quantification of immobilized protein.

BM3 activity assay. BM3 activity determination methods were based on methods described by adapted for microplates. (Tsotsou, et al., Biosensors & Bioelectronics, 17:119-131 (2002), incorporated by reference herein in its entirety.) Briefly, BM3 catalyzed the oxidation of p-NPL to form p-NP and ω-1 hydroxylauric acid (Reaction 1). Enzyme activity was measured spectrophotometrically by the increase in absorbance at 410 nm due to the formation of p-NP. (Denisov et al., Chemical Reviews, 105(6):2253-2278 (2005), incorporated herein in its entirety.) BM3 reactions were run at 21° C. for 18 h in 2 mL microcentrifuge tubes using a total reaction volume of 0.5 mL containing 100 mM pH 8.2 phosphate buffered saline (PBS), 0.25 mM p-NPL (0.25% DMSO), 0.15 mM NADPH, 1 mM magnesium chloride, 1 mM glucose, and 3.6 μg/mL CYP (˜60 nM). Free enzyme controls also contained 60 nM GDH. Immobilized BM3 was pelleted magnetically and its supernatant read for absorbance. p-NP was quantified using a linear standard curve containing 0-0.5 mM p-NP in 100 mM pH 8.2 PBS (R²>0.98). One unit (U) of BM3 activity was defined as 1 μmol p-NP generated per minute at 21° C. in 100 mM PBS (pH 8.2).

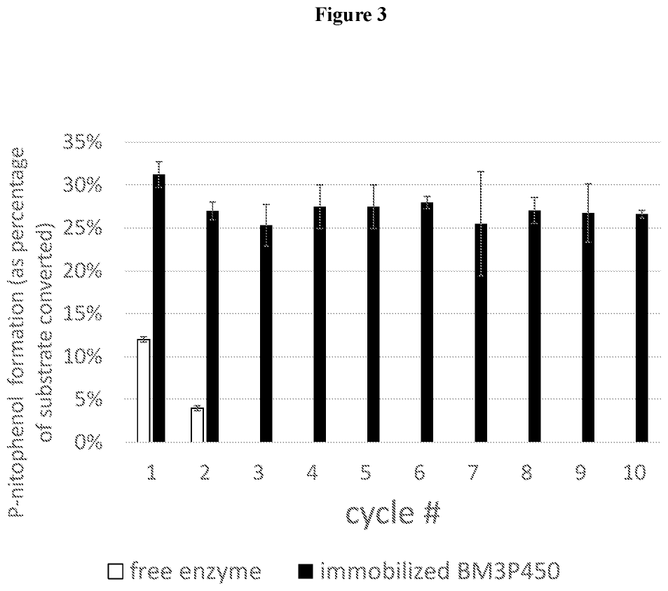

Reusability of immobilized CYP. After an activity assay was completed, CYP BMCs were pelleted magnetically and their supernatants removed for analysis. The BMCs were then rinsed with an assay's volume of cold ultrapure water. A substrate buffer was then added to BMCs to initiate a second reaction cycle. This process was repeated ten times to demonstrate reusability of CYP BM3s. (FIG. 3.) The immobilized enzyme was compared to a stock of free enzyme prepared on the same day as the immobilization, stored on ice.

Protein quantification. BMCs were pelleted magnetically and protein content in the supernatant was determined using the Bradford method, including a linear BSA standard curve (R²>0.99). (Bradford, Analytical Biochemistry, 72(1-2):248-254 (1976), incorporated herein by reference in its entirety.)

Results

BNCs showed similar activity to free enzyme when BM3 was co-immobilized with glucose dehydrogenase (GDH, for cofactor regeneration), catalase and superoxide dismutase (CAT/SOD, for ROS control) and NADPH (for improved stability during immobilization). The optimized immobilized BM3 displayed >99% activity relative to the free enzyme for the formation of p-nitrophenol as the oxidation product of p-nitrophenyl laurate. BM3+SS was immobilized with >99% immobilization yield with a total loading of 2.5% and a CYP loading of 0.3%. Controls showed that uncatalyzed p-NP formation only reached 2% conversion after 18 h. Immobilized enzyme with complete SS had 25% conversion whereas the free enzyme only reached 16%. Omission of NADPH and ROS control from the immobilization lowered conversion to only 10%. Inclusion of ROS control without NADPH resulted in 14% conversion (FIG. 3). These results showed that both ROS control and NADPH improve activity of immobilized BM3. BM3+SS demonstrated consistent activity for 10 cycles of p-NPL oxidation. Activity was stable at about 25% conversion under standard conditions. Free enzyme conversion from the initial stock (stored at 4° C.) dropped to 4% by the second day. By the third day, free enzyme conversion was equivalent to the baseline uncatalyzed oxidation rate of p-NPL indicating that all activity was lost.

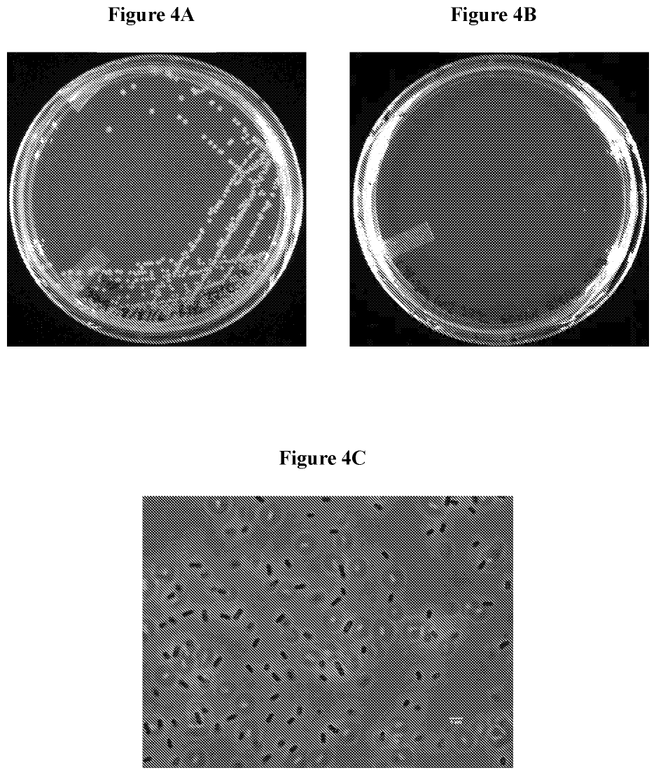

Unexpectedly, over time, bacteria grew in reactions containing the free BM3 crude extracts but not the immobilized extracts. A more concentrated stock of free BM3 appeared turbid after 24 h on ice. A 10 μL sterile loop was used to inoculate an LB agar plate. Small beige colonies (1-2 mm) appeared after 24 h incubation of the plate at 37° C. These colonies were confirmed to be formed due to an isolated rod-shaped bacterium, possibly the expression host for BM3. When a similar inoculum was prepared using the supernatant of immobilized BM3, no colonies developed (FIG. 4) This shows that the immobilization impeded growth of potential bacterial contaminants from the crude enzyme preparation or from external sources. The system is not thought to be bactericidal but it is hypothesized that bacterial growth is reduced because proteins and enzymes are entrapped in the BNCs and not available to bacteria.

### Example 2

### Human Cytochrome p450 with Glucose-6-phosphate Dehydrogenase, Catalase, Superoxide Dismutase, and NADPH Co-Immobilization on Magnetic Supports

Magnetically-immobilized P450 activity and recyclability. BNCs containing recombinant human CYPs (MW=56-58 kDa) are prepared. Endoplasmic reticulum near cytochrome P450 reductase (CPR) is expressed with or without cytochrome b5. Magnetite nanoparticles are prepared with about 20% loading, then templated onto ground magnetic macroporous polymeric hybrid scaffolds, resulting in projected final loadings on BMCs above 0.1% CYP loading). Metabolic competence is evaluated for yields and metabolite profiles. CYP3A4 activity is determined on terfenadine. CYP1A2 activity is determined on phenacetin. CYP2B6 activity is determined on bupropion. A mixed human CYP system is also evaluated for metabolic competence. Metabolites from metabolic competence studies are used to generate concentration-response curves for cytotoxicity on human embryonic kidney cells.

Materials and Equipment. HEK293 cells, Trypsin-EDTA buffer, Dulbecco's minimal essential medium (DMEM), and fetal bovine serum come from ATCC (Manassas, Va.). Corning® Supersomes™ Human CYP+Oxidoreductase+b5 3A4, 1A2, 2B6, and 2E1 (without b5) are purchased from Corning (Corning, N.Y.). ATP-quantitation assay kit (CellTiter-Glo) is purchased from Promega (Madison, Wis.). Bovine serum albumin (BSA), Bovine liver catalase (CAT), Bovine erythrocyte cytosolic superoxide dismutase (SOD) expressed in E. coli, glucose (beta-d-glucose), p-nitrophenyl laurate (p-NPL), p-nitrophenol (p-NP), nicotinamide adenine dinucleotide phosphate (reduced) tetrasodium salt (NADPH), penicillin, streptomycin, glucose-6-phosphate, glucose-6 phosphate dehydrogenase (G6PDH), ethoxyresorufin, resorufin, coumarin, 7-hydroxycoumarin, terfenadine, hydroxyterfenadine, phenacetin, acetaminophen, bupropion, and 1-hydroxybupropion are purchased from Sigma-Aldrich (St. Louis, Mo., USA). Dimethyl sulfoxide (DMSO) is purchased from Fisher Scientific (Fair Lawn, N.J., USA). Hydrochloric acid, sodium hydroxide, magnesium chloride, and phosphate buffer salts are from Macron Fine Chemicals (Center Valley, Pa., USA). The Quick Start™ Bradford Protein Assay is purchased from Bio-Rad (Hercules, Calif., USA). Stock solutions are made with 18.2 MΩ-cm water purified by Barnstead™ Nanopure™. Absorbance is measured in triplicate in Costar™ 3635 UV-transparent microplates using Biotek Synergy4198 plate reader operated with Gen5™ software. Fluorescence is measured in Costar™ 3574 black-bottom microplates. Luminescence is measured in opaque white tissue-culture treated multi-well microplates Greiner Bio-One North America (Monroe, N.C.). A sonicator (FB-505) with ⅛″ probe is purchased from Fisher Scientific® (Waltham, Mass.). ZymTrap™, (powder, 100-500 μm, MO32-40, Zymtronix, Ithaca N.Y.) was use as magnetic scaffold for the immobilized enzyme systems of P450s.

Reagents. All aqueous stocks are prepared with ultrapure (MQ) water. Lyophilized Corning® Supersomes™, G6PDH, and NADPH are dissolved in ice-cold oxygen free 50 mM TRIS HCl, pH 7.5 and prepared fresh daily. Ethoxyresorufin, resorufin, coumarin, and 7-hydroxycoumarin, terfenadine stock solutions are prepared in pure DMSO to 100 mM and stored at 4° C. Magnesium chloride (1M), glucose (100 mM), and glucose-6-phosphate (100 mM) are dissolved in water and stored at 4° C. All stock solutions are kept on ice. Dilutions are made just before use in assays and allowed to equilibrate to room temperature (21° C.).

Tissue Culture. HEK293 cells are cultured following the procedures used by Xia et al., Environmental Health Perspectives, 116(3):284-291 (2008), incorporated by reference herein in its entirety.

Immobilization. Supersome immobilizations are optimized using the methods taught in Int'l Pub. Nos. WO2012122437 and WO2014055853, U.S. Prov. App. No. 62/323,663, and Corgié et al., Chemistry Today, 34:15-20 (2016). The foregoing are incorporated by reference herein in their entirety. The non-CYP biological and chemical components of the immobilization as follows are referred to as the CYP Support System (SS): G6PDH for cofactor regeneration, CAT/SOD for reactive oxygen species (ROS) control, and NADPH for stability during immobilization. Free G6PDH)/CAT/SOD/NADPH stock (500 μg/mL CYP, 100:100:1:1:100 molar ratios) are prepared in cold buffer using fresh enzyme stocks. A 5 mL 2500 μg/ml MNP stock is sonicated at the 40% amplitude for 1 min, equilibrated to room temperature using a water bath, and its pH is adjusted to 3. Free CYP+SS (500 μL) is dispensed into a 2 mL microcentrifuge tube to which an equal volume of sonicated MNPs is added, then pipette mixed 10 times. CYP+SS BMCs are prepared by adding 1 mL of BNCs to 98.75 mg MO32-40 ZymTrap powder and pipette mixing 10 times. These BMCs are gently mixed on a rotator for 1 h, then were pelleted magnetically. Their supernatants were saved for quantification of immobilized protein using the Bradford method and NADPH using its molar absorptivity at 340 nm (ε=6.22 mM⁻¹cm⁻¹).

Supersome immobilization screening and activity assays. Supersome CYPs optimal immobilization condition is determine through a two-phase screening in microplates following the methods of Corgié (2016) with some modifications. The initial screening determines the combination of MNP pH and enzyme buffer concentration that results in the highest activity and the highest immobilization yields. The second phase optimizes the concentration of MNP. The optimal immobilization conditions determined for CYP3A4 are applied to the other human CYPs and mixed human CYP systems. The activity assays used for screening measure a change in fluorescence due to either the conversion of ethoxyresorufin to resorufin (dealkylation activity) or the conversion of coumarin to 7-hydroxycoumarin (hydroxylation activity). Supersome™ reactions are run at 37° C. for 18 h in 2 mL microcentrifuge tubes with a total reaction volume of 0.15 mL containing 100 mM pH 7.4 phosphate buffered saline (PBS), 0.05 mM substrate (0.05% DMSO), 0.15 mM NADPH, 1 mM magnesium chloride, 1 mM glucose-6-phosphate, and 20 nM CYP. Free enzyme controls also contain 200 nM G6PDH. Immobilized Supersomes are pelleted magnetically and their supernatants read for fluorescence intensity. Resorufin and 7-hydroxycoumarin excitation/emission wavelengths are 530/580 nm and 370/450 nm respectively. Reaction products are quantified using a linear standard curve containing 0-0.1 mM product in 100 mM pH 7.4 PBS with 0.05% DMSO. One unit (U) of CYP dealkylation activity is defined as 1 μmol resorufin generated per minute at 37° C. in 100 mM PBS. One unit (U) of CYP dealkylation activity is defined as 1 μmol resorufin generated per minute at 37° C. in 100 mM PBS. One unit (U) of CYP hydroxylation activity is defined as 1 μmol 7-hydroxycoumarin generated per minute at 37° C. in 100 mM PBS.

Metabolic competence is a metric that compares the metabolite profiles and yields of immobilized CYPs with their non-immobilized analogs. Using the optimized immobilized human CYPs+SS, the metabolic competence of these systems is evaluated using CYP3A4 activity on terfenadine, CYP1A2 activity on phenacetin, and CYP2B6 activity on bupropion. A mixed human CYP system is also evaluated for metabolic competence. The activities above are measured using HPLC analysis of reaction supernatants. Separate reactions are run at 37° C. for 30 min and 18 h in fluorescence black-bottom microplates with a total reaction volume of 0.15 mL (triplicates) containing 100 mM pH 7.4 phosphate buffered saline (PBS), 0.05 mM substrate (0.05% DMSO), 0.15 mM NADPH, 1 mM magnesium chloride, 1 mM glucose-6-phosphate, and 200 nM CYP. Free enzyme controls also contain 200 nM G6PDH at the designated endpoints, 30 μL of supernatant is saved and frozen at −80° C. and another 30 μL is transferred into 60 μL acetonitrile and frozen at −80° C. for HPLC analysis. The acetonitrile free sample is diluted 1:200, 1:400, 1:800, 1:1600, 1:3200, 1:6400, 1:12800, 1:25600 in 100 mM PBS pH 7.4 and saved for cell viability assays.

Cell viability assay. The ATP-quantitation-based cell viability assay is taught by Xia (2008). It is used to assess a metabolite concentration-response (i.e. cytotoxicity).

Protein quantification. BMCs are pelleted magnetically and protein content in the supernatant is determined using the Bradford method and a linear BSA standard curve (R²>0.99). (Bradford, Analytical Biochemistry, 72(1-2):248-254 (1976), incorporated herein by reference in its entirety.)

Results

Optimized immobilized human CYPs+SS demonstrate metabolic competence by achieving overlapping metabolite profiles and yields (from HPLC analysis) and similar dose-response curves as their non-immobilized counterparts. Metabolic competence may be observed for both the single CYP and a mixed CYP systems.

### Example 3

### Magnetic Mixer for the Use of Immobilized Oxidative Enzymes in High-Throughput Microplate Format

Cytochromes P450 require molecular dioxygen. Initial modeling have shown that dioxygen can become limiting for substrate concentrations above 240 μM at 37° C. Moreover a significant portion of the O₂ (30% or more) is converted to ROS which reduces the effective concentration of dissolved O₂ for substrate oxidation. Finally, local consumption of O₂ during the reaction can result in O₂ depleted volumes or O₂ concentration gradients—particularly if the enzymes are immobilized and used as heterogeneous catalysts. In the case of gradients, the concentration of dioxygen is highest at the air/liquid interface. Mixing is hence required to ensure homogenous and non-limiting concentration of dioxygen.

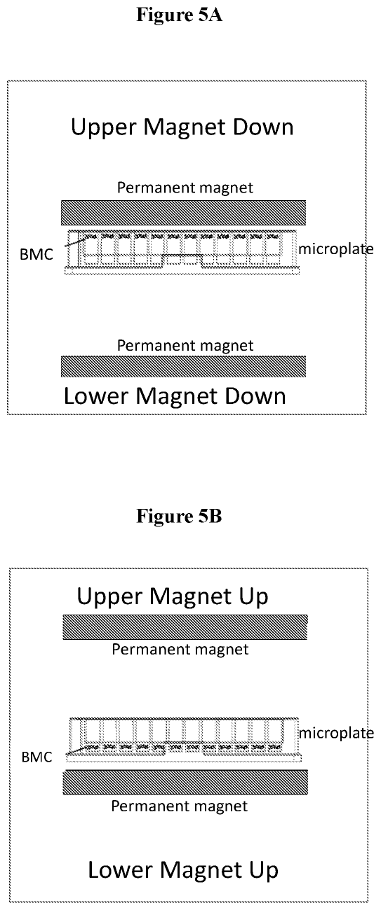

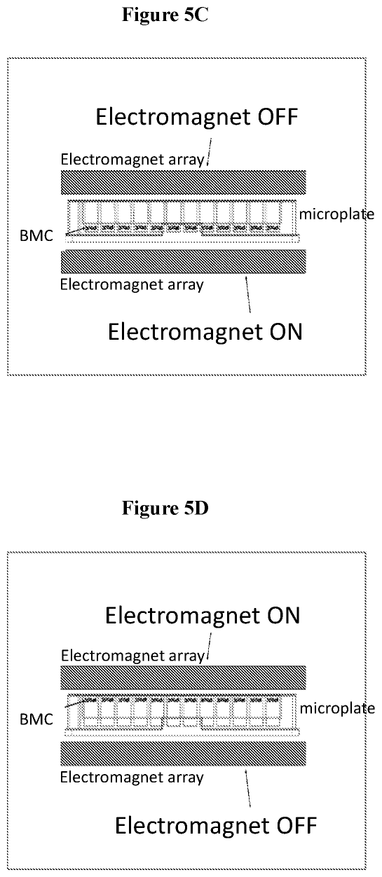

Homogenous mixing in microplates is performed via shaking or micro-stirring bars. Alternatively, to ensure non-limiting concentration of dioxygen for the use of immobilized P450 enzyme systems in a microplate format, a magnetic mixing apparatus was designed and built. The goal was to bounce the magnetically immobilized enzymes vertically (FIGS. 5A-5D) and use the motion of the particles to mix the reaction volume from the air/liquid interface to the bottom of the well. The prototype used two arrays of neodymium magnets 5″×4″×⅛″ each, spaced 3″ apart to avoid any magnetic interaction between the arrays. The arrays were placed in 3D printed carriers and attached to lead screws coupled to stepper motors for vertical movement. A microplate and holding tray was mounted in between the arrays and connected to a lead screw and stepper motor. The tray moved horizontally to provide sufficient clearance to easily place and remove the microplate. Although the arrays' maximum travel distance was 3″, the length of the gap, a distance of 0.75″, was found to sufficiently bounce the magnetic catalysts. The motors were controlled by a microcontroller and motor driver. The microcontroller received commands from the user and forwarded them to the motor driver. The motor driver, connected to a power supply, provided sufficient voltage and current to power the motors. Movement commands were uploaded to the microcontroller either individually or as a script. The commands comprised a list of commands that were executed sequentially. Individual commands were used for calibration while scripts automated the movement of the magnetic arrays. The motor speed, and consequently the period of oscillation, was controllable through the microcontroller.

In some embodiments, the magnetic incubation mixer is a fully enclosed system designed to process microplates. The primary components are the incubation chamber, magnetic arrays, heating control system, and pipetting-transfer head. The microplate is placed on a tray which retracts inside the incubator. The incubator is lined with insulation to effectively maintain the temperature regulated by the heating control system. The incubator also contains magnetic arrays, constructed with either electromagnets or permanent magnets, and the heating system. The arrays are used to move the magnetic material inside the microplate wells. If using electromagnets, arrays of electromagnets are mounted flush with the top and bottom faces of the microplate. The power delivered to the arrays is alternated to move the magnetic material vertically. If using permanent magnets, arrays of magnets are mounted above and below the microplate at a set vertical distance apart. The gap between the arrays always remains the same. The arrays are moved up and down repeatedly allowing the magnetic field from the arrays to move the magnetic material. During the mixing process, the ambient temperature is raised to the incubation temperature set by the user. The temperature is controlled using a temperature sensor, heater, and feedback loop. The sensor detects the internal ambient temperature and transmits the reading to the feedback loop. The feedback loop is responsible for maintaining a steady temperature inside the incubation chamber and controls the amount of power delivered to the heater based on the temperature reading and the desired temperature. Once magnetic processing is complete, the plate is ejected from the incubator. An integrated pipetting station transfers the supernatant to an alternate microplate, leaving only the magnetic material. Permanent magnets located beneath the tray ensure that the magnetic materials are not inadvertently transferred with the supernatant.

### Exemplary Sequences

Bifunctional P450/NADPH-P450
reductase [Bacillus megaterium]
SEQ ID NO: 1
MTIKEMPQPKTFGELKNLPLLNTDKPVQALMKIADELGEIFKFEAPGRVT
RYLSSQRLIKEACDESRFDKNLSQALKFVRDFAGDGLFTSWTHEKNWKKA
HNILLPSFSQQAMKGYHAMMVDIAVQLVQKWERLNADEHIEVPEDMTRLT
LDTIGLCGFNYRFNSFYRDQPHPFITSMVRALDEAMNKLQRANPDDPAYD
ENKRQFQEDIKVMNDLVDKIIADRKASGEQSDDLLTHMLNGKDPETGEPL
DDENIRYQIITFLIAGHETTSGLLSFALYFLVKNPHVLQKAAEEAARVLV
DPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRA
CIGQQFALHEATLVLGMMLKHFDFEDHTNYELDIKETLTLKPEGFVVKAK
SKKIPLGGIPSPSTEQSAKKVRKKAENAHNTPLLVLYGSNMGTAEGTARD
LADIAMSKGFAPQVATLDSHAGNLPREGAVLIVTASYNGHPPDNAKQFVD
WLDQASADEVKGVRYSVFGCGDKNWATTYQKVPAFIDETLAAKGAENIAD
RGEADASDDFEGTYEEWREHMWSDVAAYFNLDIENSEDNKSTLSLQFVDS
AADMPLAKMHGAFSTNVVASKELQQPGSARSTRHLEIELPKEASYQEGDH
LGVIPRNYEGIVNRVTARFGLDASQQIRLEAEEEKLAHLPLAKTVSVEEL
LQYVELQDPVTRTQLRAMAAKTVCPPHKVELEALLEKQAYKEQVLAKRLT
MLELLEKYPACEMKFSEFIALLPSIRPRYYSISSSPRVDEKQASITVSVV
SGEAWSGYGEYKGIASNYLAELQEGDTITCFISTPQSEFTLPKDPETPLI
MVGPGTGVAPFRGFVQARKQLKEQGQSLGEAHLYFGCRSPHEDYLYQEEL
ENAQSEGIITLHTAFSRMPNQPKTYVQHVMEQDGKKLIELLDQGAHFYIC
GDGSQMAPAVEATLMKSYADVHQVSEADARLWLQQLEEKGRYAKDVWAG
Cytochrome P450 3A4 isoform 1 [Homo sapiens]
SEQ ID NO: 2
MALIPDLAMETWLLLAVSLVLLYLYGTHSHGLFKKLGIPGPTPLPFLGNI
LSYHKGFCMFDMECHKKYGKVWGFYDGQQPVLAITDPDMIKTVLVKECYS
VFTNRRPFGPVGFMKSAISIAEDEEWKRLRSLLSPTFTSGKLKEMVPIIA
QYGDVLVRNLRREAETGKPVTLKDVFGAYSMDVITSTSFGVNIDSLNNPQ
DPFVENTKKLLRFDFLDPFFLSITVFPFLIPILEVLNICVFPREVTNFLR
KSVKRMKESRLEDTQKHRVDFLQLMIDSQNSKETESHKALSDLELVAQSI
IFIFAGYETTSSVLSFIMYELATHPDVQQKLQEEIDAVLPNKAPPTYDTV
LQMEYLDMVVNETLRLFPIAMRLERVCKKDVEINGMFIPKGVVVMIPSYA
LHRDPKYWTEPEKFLPERFSKKNKDNIDPYIYTPFGSGPRNCIGMRFALM
NMKLALIRVLQNFSFKPCKETQIPLKLSLGGLLQPEKPVVLKVESRDGTV
SGA
Cytochrome P450 1A2 [Homo sapiens]
SEQ ID NO: 3
MALSQSVPFSATELLLASAIFCLVFWVLKGLRPRVPKGLKSPPEPWGWPL
LGHVLTLGKNPHLALSRMSQRYGDVLQIRIGSTPVLVLSRLDTIRQALVR
QGDDFKGRPDLYTSTLITDGQSLTFSTDSGPVWAARRRLAQNALNTFSIA
SDPASSSSCYLEEHVSKEAKALISRLQELMAGPGHFDPYNQVVVSVANVI
GAMCFGQHFPESSDEMLSLVKNTHEFVETASSGNPLDFFPILRYLPNPAL
QRFKAFNQRFLWFLQKTVQEHYQDFDKNSVRDITGALFKHSKKGPRASGN
LIPQEKIVNLVNDIFGAGFDTVTTAISWSLMYLVTKPEIQRKIQKELDTV
IGRERRPRLSDRPQLPYLEAFILETFRHSSFLPFTIPHSTTRDTTLNGFY
IPKKCCVFVNQWQVNHDPELWEDPSEFRPERFLTADGTAINKPLSEKMML
FGMGKRRCIGEVLAKWEIFLFLAILLQQLEFSVPPGVKVDLTPIYGLTMK
HARCEHVQARLRFSIN
CYP2D6 [Homo sapiens]
SEQ ID NO: 4
MGLEALVPLAMIVAIFLLLVDLMHRRQRWAARYPPGPLPLPGLGNLLHVD
FQNTPYCFDQLRRRFGDVFSLQLAWTPVVVLNGLAAVREALVTHGEDTAD
RPPVPITQILGFGPRSQGRPFRPNGLLDKAVSNVIASLTCGRRFEYDDPR
FLRLLDLAQEGLKEESGFLREVLNAVPVLLHIPALAGKVLRFQKAFLTQL
DELLTEHRMTWDPAQPPRDLTEAFLAEMEKAKGNPESSFNDENLCIVVAD
LFSAGMVTTSTTLAWGLLLMILHPDVQRRVQQEIDDVIGQVRRPEMGDQA
HMPYTTAVIHEVQRFGDIVPLGVTHMTSRDIEVQGFRIPKGTTLITNLSS
VLKDEAVWEKPFRFHPEHFLDAQGHFVKPEAFLPFSAGRRACLGEPLARM
ELFLFFTSLLQHFSFSVPTGQPRPSHHGVFAFLVTPSPYELCAVPR
Cytochrome P450-2E1 [Homo sapiens]
SEQ ID NO: 5
MSALGVTVALLVWAAFLLLVSMWRQVHSSWNLPPGPFPLPIIGNLFQLEL
KNIPKSFTRLAQRFGPVFTLYVGSQRMVVMHGYKAVKEALLDYKDEFSGR
GDLPAFHAHRDRGIIFNNGPTWKDIRRFSLTTLRNYGMGKQGNESRIQRE
AHFLLEALRKTQGQPFDPTFLIGCAPCNVIADILFRKHFDYNDEKFLRLM
YLFNENFHLLSTPWLQLYNNFPSFLHYLPGSHRKAIKNVAEVKEYVSERV
KEHHQSLDPNCPRDLTDCLLVEMEKEKHSAERLYTMDGITVTVADLFFAG
TETTSTTLRYGLLILMKYPEIEEKLHEEIDRVIGPSRIPAIKDRQEMPYM
DAVVHEIQRFITLVPSNLPHEATRDTIFRGYLIPKGTVVVPTLDSVLYDN
QEFPDPEKFKPEHFLNENGKFKYSDYFKPFSTGKRVCAGEGLARMELFLL
LCAILQHFNLKPLVDPKDIDLSPIHIGFGCIPPRYKLCVIPRS
Cytochrome P450-2E1 [Homo sapiens]
SEQ ID NO: 6
MSALGVTVALLVWAAFLLLVSMWRQVHSSWNLPPGPFPLPIIGNLFQLEL
KNIPKSFTRLAQRFGPVFTLYVGSQRMVVMHGYKAVKEALLDYKDEFSGR
GDLPAFHAHRDRGIIFNNGPTWKDIRRFSLTTLRNYGMGKQGNESRIQRE
AHFLLEALRKTQGQPFDPTFLIGCAPCNVIADILFRKHFDYNDEKFLRLM
YLFNENFHLLSTPWLQLYNNFPSFLHYLPGSHRKAIKNVAEVKEYVSERV
KEHHQSLDPNCPRDLTDCLLVEMEKEKHSAERLYTMDGITVTVADLFFAG
TETTSTTLRYGLLILMKYPEIEEKLHEEIDRVIGPSRIPAIKDRQEMPYM
DAVVHEIQRFITLVPSNLPHEATRDTIFRGYLIPKGTVVVPTLDSVLYDN
QEFPDPEKFKPEHFLNENGKFKYSDYFKPFSTGKRVCAGEGLARMELFLL
LCAILQHFNLKPLVDPKDIDLSPIHIGFGCIPPRYKLCVIPRS
Cytochrome P450, family 2,
subfamily C, polypeptide 9 [Homo sapiens]
SEQ ID NO: 7
MDSLVVLVLCLSCLLLLSLWRQSSGRGKLPPGPTPLPVIGNILQIGIKDI
SKSLTNLSKVYGPVFTLYFGLKPIVVLHGYEAVKEALIDLGEEFSGRGIF
PLAERANRGFGIVFSNGKKWKEIRRFSLMTLRNFGMGKRSIEDRVQEEAR
CLVEELRKTKASPCDPTFILGCAPCNVICSIIFHKRFDYKDQQFLNLMEK
LNENIKILSSPWIQICNNFSPIIDYFPGTHNKLLKNVAFMKSYILEKVKE
HQESMDMNNPQDFIDCFLMKMEKEKHNQPSEFTIESLENTAVDLFGAGTE
TTSTTLRYALLLLLKHPEVTAKVQEEIERVIGRNRSPCMQDRSHMPYTDA
VVHEVQRYIDLLPTSLPHAVTCDIKFRNYLIPKGTTILISLTSVLHDNKE
FPNPEMFDPHHFLDEGGNFKKSKYFMPFSAGKRICVGEALAGMELFLFLT
SILQNFNLKSLVDPKNLDTTPVVNGFASVPPFYQLCFIPV

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1. A composition comprising self-assembled mesoporous aggregates of magnetic nanoparticles and
a. a first enzyme requiring a diffusible cofactor having a first enzymatic activity;
b. a second enzyme comprising a cofactor regeneration activity;
wherein said cofactor is utilized in said first enzymatic activity; wherein said first and second enzymes are magnetically-entrapped within said mesopores formed by said aggregates of magnetic nanoparticles and said first and second enzymes function by converting a diffusible substrate into a diffusible product.

2. The composition of claim 1, wherein said co-factor is entrapped in said mesoporous aggregates of magnetic nanoparticles with said first and second enzymes.

3. The composition of claim 1, wherein said mesoporous aggregates of magnetic nanoparticles have an iron oxide composition.

4. The composition of claim 1, wherein said mesoporous aggregates of magnetic nanoparticles have a magnetic nanoparticle size distribution in which at least 90% of magnetic nanoparticles have a size of at least 3 nm and up to 30 nm, and an aggregated particle size distribution in which at least 90% of said mesoporous aggregates of magnetic nanoparticles have a size of at least 10 nm and up to 500 nm.

5. The composition of claim 1, wherein said mesoporous aggregates of magnetic nanoparticles possess a saturated magnetization of at least 10 emu/g.

6. The composition of claim 5, wherein said mesoporous aggregates of magnetic nanoparticles possess a remanent magnetization up to 5 emu/g.

7. The composition of claim 1, wherein said first and second enzymes are contained in said mesoporous aggregates of magnetic nanoparticles in up to 100% of saturation capacity.

8. The composition of claim 1, wherein said first and second enzymes are physically inaccessible to microbes.

9. The composition of claim 1, wherein said first enzyme is an oxidative enzyme.

10. The composition of claim 9, wherein said oxidative enzyme is a Flavin-containing oxygenase; wherein said composition further comprises a third enzyme having a co-factor reductase activity that is co-located with said first enzyme.

11. The composition of claim 9, wherein said oxidative enzyme is a P450 monooxygenase; wherein said composition further comprises a third enzyme having a co-factor reductase activity that is co-located with said first enzyme.

12. The composition of claim 11, wherein said P450 monooxygenase and said third enzyme are comprised within a single protein.

13. The composition of claim 12, wherein said single protein comprises a bifunctional cytochrome P450/NADPH—P450 reductase.

14. The composition of claim 12, wherein said single protein has BM3 activity and has at least a 90% sequence identity to SEQ ID NO:1.

15. The composition of claim 11, wherein said P450 monooxygenase is co-located with said third enzyme within a lipid membrane.

16. The composition of claim 11, wherein said third enzyme is a cytochrome P450 reductase.

17. The composition of claim 15, wherein said P450 monooxygenase comprises a P450 sequence that is mammalian.

18. The composition of claim 17, wherein said P450 monooxygenase comprises a P450 sequence that is human.

19. The composition of claim 18, wherein said P450 monooxygenase comprises CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1,CYP3A4, CYP3A5, CYP3A7, CYP3A43,CYP4A11, CYP4A22, CYP4B1, CYP4F2, CYP4F3, CYP4F8, CYP4F11, CYP4F12, CYP4F22, CYP4V2, CYP4X1, CYP4Z1,CYP5A1,CYP7A1, CYP7B1,CYP8A1, CYP8B1,CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1, or CYP51A1.

20. The composition of claim 17, wherein said P450 monooxygenase comprises a P450 sequence that is of an origin selected from the group consisting of primate, mouse, rat, dog, cat, horse, cow, sheep, and goat.

21. The composition of claim 15 wherein said P450 monooxygenase comprises a P450 sequence that is of an origin selected from the group consisting of insect, fish, fungus, yeast, protozoan, and plant.

22. The composition of claim 1, wherein said second enzyme is selected from the group consisting of a carbonyl reductase, an aldehyde dehydrogenase, an aryl-alcohol dehydrogenase, an alcohol dehydrogenase, a pyruvate dehydrogenase, a D-1 xylose dehydrogenase, an oxoglutarate dehydrogenase, an isopropanol dehydrogenase, a glucose-6-phosphate dehydrogenase, a glucose dehydrogenase, a malate dehydrogenase, a formate dehydrogenase, a benzaldehyde dehydrogenase, a glutamate dehydrogenase, and an isocitrate dehydrogenase.

23. The composition of claim 1, wherein said cofactor is nicotinamide adenine dinucleotide+hydrogen (NADH), nicotinamide adenine dinucleotide phosphate+hydrogen (NADPH), Flavin adenine dinucleotide+hydrogen (FADH), or glutathione.

24. The composition of claim 11, further comprising a fourth enzyme that reduces a reactive oxygen species (ROS).

25. The composition of claim 24, wherein said fourth enzyme is a catalase, a superoxide dismutase (SOD), or a glutathione peroxidase/glutathione-disulfide reductase.

26. The composition of claim 1, wherein said first enzyme participates in phase I metabolism.

27. The composition of claim 24, further comprising a fifth enzyme that participates in phase II or phase III metabolism.

28. The composition of claim 27, wherein said fifth enzyme is a UDP-glucoronosyl transferase, a sulfotransferase, a monoamine oxidase, or a carboxylesterase.

29. The composition of claim 1 any one of claims 1-28, wherein said composition of mesoporous aggregates are assembled onto a macroporous magnetic scaffold.

30. The composition of claim 29, wherein said macroporous magnetic scaffold is a polymeric hybrid scaffold comprising a cross-linked water-insoluble polymer and an approximately uniform distribution of embedded magnetic microparticles (MMP).

31. The composition of claim 30, wherein said magnetic macroporous polymeric hybrid scaffold comprises PVA and a polymer selected from the group consisting of CMC, alginate, HEC, and EHEC.

32. The composition of claim 1, wherein one or more said enzymes are produced by recombinant DNA technology.

33. The composition of claim 1, wherein one or more said enzymes are produced by cell-free protein synthesis.

34. A method of manufacturing a chemical, comprising exposing the composition of claim 1 to said diffusible substrate in a first reaction.

35. The method of claim 34, further comprising the step of magnetically mixing said first reaction.

36. The method of claim 34, further comprising recovering said diffusible product.

37. The method of claim 34, further comprising the step of magnetically recovering said composition from other components of said first reaction.

38. The method of claim 37, further comprising the step of exposing said composition to a second reaction.

39. The method of claim 38, further comprising recovering said diffusible product from said second reaction.

40. The method of claim 34, wherein said first reaction is a batch reaction.

41. The method of claim 40, wherein said batch reaction is in a microplate.

42. The method of claim 34, wherein said first reaction is a packed bed reaction.

43. The method of claim 34, wherein said first reaction is a continuous flow reaction.

44. The composition of claim 11, further comprising a fourth enzyme that reduces a reactive oxygen species (ROS).

45. The composition of claim 44, wherein said fourth enzyme is a catalase, a superoxide dismutase (SOD), or a glutathione peroxidase/glutathione-disulfide reductase.

46. The composition of 44, further comprising a fifth enzyme that participates in phase II or phase III metabolism.

47. The composition of claim 46 wherein said fifth enzyme is a UDP-glucoronosyl transferase, a sulfotransferase, a monoamine oxidase, or a carboxylesterase.
